(19) United States
(12) Patent Application Publication
(10) Pub. No.: US 2016/0024532 A1
(43) Pub. Date: Jan. 28, 2016

(54) ATP DRIVEN DIRECT PHOTOSYNTHETIC PRODUCTION OF FUELS AND CHEMICALS

(71) Applicant: The Regents of the University of California, Oakland, CA (US)

(72) Inventors: James C. Liao, Los Angeles, CA (US); Ethan I. Lan, Los Angeles, CA (US)

(21) Appl. No.: 14/380,051

(22) PCT Filed: Feb. 23, 2013
(86) PCT No.: PCT/US13/27533

§ 371 Date: Aug. 20, 2014

Related U.S. Application Data
(60) Provisional application No. 61/602,273, filed on Feb. 23, 2012.

(51) Int. Cl. C12P 7/16; C12N 9/10; C12N 9/00

(52) U.S. Cl. CPC C12P 7/16; C12N 9/93; C12N 9/1029; C12Y 203/01; C12Y 203/01194; C12Y 604/01002

(57) ABSTRACT

Provided herein are metabolically-modified microorganisms useful for producing biofuels. More specifically, provided herein are methods of producing high alcohols including isobutanol, 1-butanol, 1-propanol, 2-methyl-l-butanol, 3-methyl-1-butanol and 2-phenylethanol from a suitable substrate.

### CROSS REFERENCE TO RELATED APPLICATIONS

This application claims priority to U.S. Provisional Application No. ______, filed Feb. 23, 2012, the disclosure of which is incorporated herein by reference in its entirety.

### BACKGROUND

According to the US Energy Information Administration (EIA, 2007), world energy-related CO₂ emissions in 2004 were 26,922 million metric tons and increased 26.7% from 1990. As a result, atmospheric levels of CO₂ have increased by about 25% over the past 150 years. Thus, it has become increasingly important to develop new technologies to reduce CO₂ emissions.

The world is also facing costly gas and oil and limited reserves of these precious resources. Biofuels have been recognized as an alternative energy source. While efforts have been made to improve various production, further developments are needed.

### SUMMARY

Recycling CO₂ into 1-Butanol, an important chemical feedstock and potential fuel, is an attractive strategy for tackling energy and environmental problems. The Coenzyme A (CoA) dependent pathway for the production of 1-butanol is the most energy efficient. While most efficient, this pathway may not be suitable for all organisms under all conditions. The first step of the CoA pathway, condensation of two acetyl-CoA, is strongly thermodynamically unfavorable. Production of 1-butanol from CO₂ by CoA pathway using engineered cyanobacteria Synechococcus elongatus PCC 7942 requires anoxic treatment with photosystem II inhibition. Contrary to the conventional wisdom that energy efficiency is crucial to microbial production, the disclosure demonstrates that ATP consumption is beneficial for the direct photosynthetic production of 1-butanol from S. elongatus PCC 7942. Energy from ATP hydrolysis was incorporated into the CoA pathway to overcome the high thermodynamic barrier for biosynthesis of acetoacetyl-CoA, the first pathway intermediate. ATP activation of acetyl-CoA into malonyl-CoA and the subsequent decarboxylative carbon chain elongation mechanism found in fatty acid and polyketide synthesis was used to irreversibly drive the synthesis of acetoacetyl-CoA. By designing a novel malonyl-CoA dependent 1-butanol production pathway, direct photosynthetic production of 1-butanol from CO₂ was obtained. In addition, the disclosure demonstrates the substitution of bifunctional aldehyde/alcohol dehydrogenase (AdhE2) with separate butyraldehyde dehydrogenase (Bldh) and alcohol dehydrogenase (YqhD) increases the 1-butanol production by 400%.

Biological production of chemical and fuel is an attractive direction towards sustainable future. In particular, 1-butanol has received increasing attention as it is a potential fuel substitute and a chemical feedstock. 1-Butanol can be produced by two distinctive pathways: 2-ketoacid pathway and Coenzyme A (CoA) dependent pathway. The 2-ketoacid pathway utilizes either threonine synthetic pathway or citramalate pathway for producing 2-ketobutyrate. Leucine biosynthesis then elongates 2-ketobutyrate into 2-ketovalarate. 2-Ketovalarate is then decarboxylated and reduced into 1-butanol. On the other hand, the CoA pathway follows the chemistry of , β-oxidation in reverse. Acetyl-CoA is condensed into acetoacetyl-CoA which is then further reduced to 1-butanol. Furthermore, using this reversed β-oxidation, 1-butanol can be elongated to 1-hexanol and other long even-numbered chain primary alcohols. A comparison of these 1-butanol synthesis pathways reveals that CoA pathway is the most carbon energy efficient pathway for producing 1-butanol. Citramalate pathway requires an additional acetyl-CoA and threonine pathway requires two ATP.

The CoA pathway is a natural fermentation pathway used by Clostridium species. However CoA pathway is not expressed well in recombinant chemoheterotrophs, resulting in low titer 1-butanol production ranging from 2.5 mg/L to 1,200 mg/L with sugar as the substrate. The hypothesized limiting step is the reduction of crotonyl-CoA by the butyryl-CoA dehydrogenase/electron transferring flavoprotein (Bcd/EtfAB) complex. Bcd/EtfAB complex is difficult to use in recombinant systems because of its poor expression, instability, and potential requirement for ferredoxin. This problem was overcome by replacing Bcd/EtfAB complex with trans-2-enoyl-CoA reductase (Ter). Ter expresses well and directly reduces crotonyl-CoA with NADH. This modified CoA 1-butanol pathway (FIG. 1; outlined in black) is catalyzed by five enzymes: thiolase (AtoB), 3-hydroxybutyryl-CoA dehydrogenase (Hbd), crotonase (Crt), Ter, and bifunctional aldehyde/alcohol dehydrogenase (AdhE2). In combination of expressing these enzymes and engineering NADH and acetyl-CoA accumulation as driving forces, successful recombinant 1-butanol production has been demonstrated in E. coli with high titer (15-30 g/L) and yield (70% -88% of theoretical). This result demonstrated the efficiency of the CoA pathway for 1-butanol fermentation.

A recombinant cyanobacteria strain capable of producing 1-butanol by fermenting its internal carbon storage upon anoxic treatment and photosystem II inhibition has been developed. The direct photosynthetic production was limited. This limitation may be due to a lack of significant driving force. Presumably, acetyl-CoA supply is insufficient to enable the energetically unfavorable condensation catalyzed by thiolase under non-fermentative condition. In sharp contrast to production of 1-butanol, the high flux production of isobutanol (450 mg/L) and isobutyraldehyde (1,100 mg/L) by S. elongatus PCC 7942 has a decarboxylation to drive the flux towards the products, highlighting the importance of driving force.

The disclosure provides a novel malonyl-CoA dependent 1-butanol pathway and demonstrate the direct photosynthetic production of 1-butanol from S. elongatus PCC 7942 under oxygenic condition. Contrary to the notion that energy efficiency is important for microbial production, the consumption of ATP is beneficial for cyanobacteria to produce 1-butanol. ATP hydrolysis was used to drive the formation of acetoacetyl-CoA. The release of free energy from ATP hydrolysis is used to overcome the thermodynamically unfavorable condensation of two acetyl-CoA. To incorporate energy of ATP hydrolysis into the CoA 1-butanol pathway, malonyl-CoA biosynthesis was used in combination with the decarboxylative carbon chain elongation using malonyl-CoA found in fatty acid and polyketide synthesis to irreversibly trap carbon flux into the formation of acetoacetyl-CoA. Despite the decarboxylation, condensation of malonyl-CoA and acetyl-CoA has the same carbon yield as the condensation of two acetyl-CoA catalyzed by thiolase. Furthermore, substitution of bifunctional aldehyde/alcohol dehydrogenase (AdhE2) with separate butyraldehyde dehydrogenase (Bldh) and alcohol dehydrogenase (YqhD) increased the 1-butanol production by 400%. While production of alcohols by CoA pathway is the most efficient pathway, it may not be suitable for all organisms under all conditions. Here we demonstrate that chain elongation by at the expense of an ATP may be more favorable in cyanobacteria.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1 is a schematic representation of variations of the CoA 1-butanol pathway. Enzymes of different cofactor preference are shown as different routes. The original CoA 1-butanol pathway is in black. Alternative routes to 1-butanol is in red. AtoB, thiolase; AccABCD, acetyl-CoA carboxylase, NphT7, acetoacetyl-CoA; PhaB, acetoacetyl-CoA reductase; PhaJ, (R)-specific enoyl-CoA hydratase; Hbd, 3-hydroxybutyryl-CoA; Crt, crotonase; Eg.Ter, Euglena gracilis Trans-2-enoyl-CoA reductase; Td. Ter, Treponema denticola; Ccr, crotonyl-CoA reductase; Bldh, butyraldehyde dehydrogenase; YqhD, NADP-dependent alcohol dehydrogenase; AdhE2, bifunctional alcohol/aldehyde dehydrogenase. EC, E. coli; RE, R. eutropha; CA, C. acetobutylicum; AC, A. caviae; TD, T. denticola; CS, C. saccharoperbutylacetonicum; CL190, Streptomyces sp. strain CL190; EG, Euglena gracilis; GP, guinea pig. SC, Streptomyces coelicolor.

FIG. 2 shows ATP driven synthesis of acetoacetyl-CoA. A) Thiolase (AtoB) catalyzed formation and thiolysis of acetoacetyl-CoA. Equilibrium constant for two acetyl-CoA condensation is very low. B) Malonyl-CoA driven formation of acetoacetyl-CoA by Acetoacetyl-CoA synthase (NphT7).

FIG. 3 shows engineered S. elongatus PCC 7942 strains displaying A) ability and inability to synthesize acetoacetyl-CoA from malonyl-CoA and acetyl-CoA by the expression of NphT7 and AtoB, respectively. B) negligible and favored thiolysis of acetoacetyl-CoA by expression of NphT7 and AtoB, respectively.

FIG. 4 shows production of 1-butanol under oxygenic condition enabled by expression of NphT7. A) growth rate between strains EL20 (nphT7.hbd.crt.ter.adhE2) and EL14 (atoB.hbd.crt.ter.adhE2) is nearly identical. B) 1-Butanol production time course by strain EL20. C) GC chromatogram demonstrating the production of 1-butanol by EL20 while EL14 produced only trace amount.

FIG. 5 shows production of 1-butanol and ethanol by recombinant E. coli strains JCL299 expressing CoA 1-butanol pathway with YqhD and Bldh from different organisms. In all strains, AtoB, PaaHl, Crt, and Ter were expressed. Strain expressing C. saccharoperbutylacetonicum NI-4 Bldh produced the highest amount of 1-butanol exceeding that of the strain expressing AdhE2 by nearly 3-fold. Sample was measured after 48 hours of anaerobic incubation in TB with 20 g/L glucose.

FIG. 6 shows data related to butanol production. A) 1-Butanol production by strains expressing different enzymes. Expression of nphT7 enables direct photosynthetic production of 1-butanol under oxygenic condition. Strains EL21 and EL22 expressing bldh and yqhD achieved the highest production. B) Enzymatic activities of CoA 1-butanol pathway enzymes in the corresponding engineered S. elongatus PCC7942 strains.

FIG. 7 depicts a nucleic acid sequence derived from a adhE2 gene encoding a polypeptide having alcohol dehydrogenase activity.

Like reference symbols in the various drawings indicate like elements.

### DETAILED DESCRIPTION

As used herein and in the appended claims, the singular forms “a,” “and,” and “the” include plural referents unless the context clearly dictates otherwise. Thus, for example, reference to “a microorganism” includes a plurality of such microorganisms and reference to “the polypeptide” includes reference to one or more polypeptides and equivalents thereof, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure.

The disclosure provides methods and compositions for the production of higher alcohols using a culture of microorganisms that utilizes CO2 as a carbon source. Examples of such microorganisms that utilize CO₂ as a carbon source include photoautotrophs. In some embodiments, that methods and compositions comprise a co-culture of photoautotrophs and a photoheterotroph or a photoautotroph and a microorganism that cannot utilize CO₂ as a carbon source.

Butanol is hydrophobic and less volatile than ethanol. 1-Butanol has an energy density closer to gasoline. Butanol at 85 percent strength can be used in cars without any change to the engine (unlike ethanol) and it produces more power than ethanol and almost as much power as gasoline. Butanol is also used as a solvent in chemical and textile processes, organic synthesis and as a chemical intermediate. Butanol also is used as a component of hydraulic and brake fluids and as a base for perfumes.

The native producers of 1-butanol, such as Clostridium acetobutylicum, also produce byproducts such as acetone, ethanol, and butyrate as fermentation products. However, these microorganisms are relatively difficult to manipulate. Genetic manipulation tools for these organisms are not as efficient as those for user-friendly hosts such as E. coli and Sarcomyces sp. and physiology and their metabolic regulation are much less understood, prohibiting rapid progress towards high-efficiency production.

The disclosure provides organisms comprising metabolically engineered biosynthetic pathways that utilize an organism's CoA pathway. Biofuel production utilizing the organism's CoA pathway offers several advantages. Not only does it avoid the difficulty of expressing a large set of foreign genes but it also minimizes the possible accumulation of toxic intermediates.

In one embodiment, the disclosure provides a recombinant microorganism comprising elevated expression of at least one target enzyme as compared to a parental microorganism or encodes an enzyme not found in the parental organism. In another or further embodiment, the microorganism comprises a reduction, disruption or knockout of at least one gene encoding an enzyme that competes with a metabolite necessary for the production of a desired higher alcohol product or which produces an unwanted product. The recombinant microorganism produces at least one metabolite involved in a biosynthetic pathway for the production of 1-butanol. In general, the recombinant microorganisms comprises at least one recombinant metabolic pathway that comprises a target enzyme and may further include a reduction in activity or expression of an enzyme in a competitive biosynthetic pathway. The pathway acts to modify a substrate or metabolic intermediate in the production of 1-butanol. The target enzyme is encoded by, and expressed from, a polynucleotide derived from a suitable biological source. In some embodiments, the polynucleotide comprises a gene derived from a bacterial or yeast source and recombinantly engineered into the microorganism of the disclosure.

As used herein, the term “metabolically engineered” or “metabolic engineering” involves rational pathway design and assembly of biosynthetic genes, genes associated with operons, and control elements of such polynucleotides, for the production of a desired metabolite, such as an acetoacetyl-CoA or higher alcohol, in a microorganism. “Metabolically engineered” can further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate or use of a cofactor or energy source, leading to a desired pathway. A biosynthetic gene can be heterologous to the host microorganism, either by virtue of being foreign to the host, or being modified by mutagenesis, recombination, and/or association with a heterologous expression control sequence in an endogenous host cell. In one embodiment, where the polynucleotide is xenogenetic to the host organism, the polynucleotide can be codon optimized.

The term “biosynthetic pathway”, also referred to as “metabolic pathway”, refers to a set of anabolic or catabolic biochemical reactions for converting (transmuting) one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as any biomass derived sugar, but also intermediate and end product metabolites used in a pathway associated with a metabolically engineered microorganism as described herein.

The term “1-butanol” or “n-butanol” generally refers to a straight chain isomer with the alcohol functional group at the terminal carbon. The straight chain isomer with the alcohol at an internal carbon is sec-butanol or 2-butanol. The branched isomer with the alcohol at a terminal carbon is isobutanol, and the branched isomer with the alcohol at the internal carbon is tert-butanol.

Recombinant microorganisms provided herein can express a plurality of target enzymes involved in pathways for the production of 1-butanol from a suitable carbon substrate.

Accordingly, metabolically “engineered” or “modified” microorganisms are produced via the introduction of genetic material into a host or parental microorganism of choice thereby modifying or altering the cellular physiology and biochemistry of the microorganism. Through the introduction of genetic material the parental microorganism acquires new properties, e.g. the ability to produce a new, or greater quantities of, a metabolite. In an illustrative embodiment, the introduction of genetic material into a parental microorganism results in a new or modified ability to produce 1-butanol. The genetic material introduced into the parental microorganism contains gene(s), or parts of genes, coding for one or more of the enzymes involved in a biosynthetic pathway for the production of 1-butanol and may also include additional elements for the expression and/or regulation of expression of these genes, e.g. promoter sequences.

An engineered or modified microorganism can also include in the alternative or in addition to the introduction of a genetic material into a host or parental micoorganism, the disruption, deletion or knocking out of a gene or polynucleotide to alter the cellular physiology and biochemistry of the microorganism. Through the reduction, disruption or knocking out of a gene or polynucleotide the microorganism acquires new or improved properties (e.g., the ability to produced a new or greater quantities of an interacellular metabolite, improve the flux of a metabolite down a desired pathway, and/or reduce the production of undesireable by-products).

Microorganisms provided herein are modified to produce metabolites in quantities not available in the parental microorganism. A “metabolite” refers to any substance produced by metabolism or a substance necessary for or taking part in a particular metabolic process. A metabolite can be an organic compound that is a starting material (e.g., glucose or pyruvate), an intermediate (e.g., acetyl-coA) in, or an end product (e.g., 1-butanol), of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

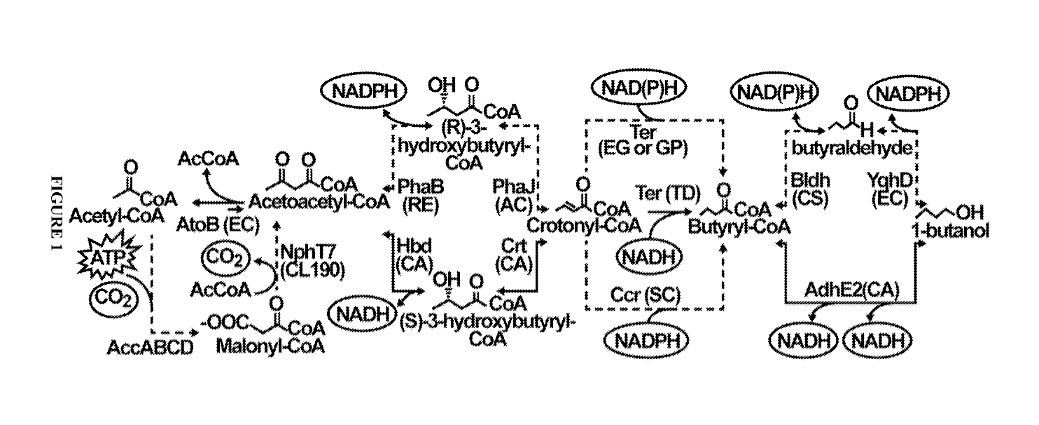

Accordingly, a recombinant microorganism provided herein includes the elevated expression of at least one target enzyme such as an enzyme that converts acetyl-CoA to malonyl-CoA, molonyl-CoA to Acetoacetyl-CoA, acetoacetyl-CoA to (R)- or (S)-3-hydroxybutyryl-CoA, (R)- or (S)-3-hydroxybutyryl-CoA to crotonyl-CoA, crotonyl-CoA to butyryl-CoA, butyryl-CoA to butyraldehyde and butyraldehyde to 1-butanol. In other embodiments, a recombinant microorganism can express a plurality of target enzymes involved in pathway to produce n-butanol as depicted in FIG. 1. The plurality of enzymes can include one or more subunits of acetyl-coA carboxylase (AccABCD, for example accession number AAC73296 AAN73296, EC 6.4.1.2), Acetoacetyl-CoA reductase (phaB, e.g., from R. eutropha) (EC 1.1.1.36) that generates 3-hydroxybutyryl-CoA from acetoacetyl-CoA and NADPH, (R)-specific enoyl-CoA hydratase (PhaJ) derived from, for example, Aeromonas caviae and Pseudomonas aeruginosa (Fukui et al., J. Bacteriol. 180:667, 1998; Tsage et al., FEMS Microbiol. Lett. 184:193, 2000), butyraldehyde dehydrogenase (Bldh) or alcohol dehydrogenase (AdhE2), Ter, Ccr, or any combination thereof.

In yet another embodiment, a recombinant microorganism provided herein includes expression or elevated expression of a crotonyl-CoA reductase as compared to a parental microorganism. The microorganism produces a metabolite that includes butyryl-CoA from a substrate that includes crotonyl-CoA. The crotonyl-CoA reductase can be encoded by a ccr gene, polynucleotide or homolog thereof. The ccr gene or polynucleotide can be derived from the genus Streptomyces.

In yet another embodiment, a recombinant microorganism provided herein includes expression or elevated expression of an alcohol dehydrogenase (ADHE2) as compared to a parental microorganism. The recombinant microorganism produces a metabolite that includes butanol from a substrate that includes butyryl-CoA. The alcohol dehydrogenase can be encoded by bdhA/bdhB polynucleotide or homolog thereof, an aad gene, polynucleotide or homolog thereof, or an adhE2 gene, polynucleotide or homolog thereof. The aad gene or adhE2 gene or polynucleotide can be derived from Clostridium acetobutylicum.

In one embodiment, the microorganism comprises a heterologous trans-2-enoyl-CoA reductase (ter). Trans-2-enoyl-CoA reductase or TER is a protein that is capable of catalyzing the conversion of crotonyl-CoA to butyryl-CoA. In certain embodiments, the recombinant microorganism expresses a TER which catalyzes the same reaction as Bcd/EtfA/EtfB from Clostridia and other bacterial species. Mitochondrial TER from E. gracilis has been described, and many TER proteins and proteins with TER activity derived from a number of species have been identified forming a TER protein family (U.S. Pat. Appl. 2007/0022497 to Cirpus et al.; Hoffmeister et al., J. Biol. Chem., 280:4329-4338, 2005, both of which are incorporated herein by reference in their entirety). A truncated cDNA of the E. gracilis gene has been functionally expressed in E. coli. This cDNA or the genes of homologues from other microorganisms can be expressed together with the n-butanol pathway genes th1, crt, adhE2, and hbd to produce n-butanol in E. coli, S. cerevisiae or other hosts.

TER proteins can also be identified by generally well known bioinformatics methods, such as BLAST. Examples of TER proteins include, but are not limited to, TERs from species such as: Euglena spp. including, but not limited to, E. gracilis, Aeromonas spp. including, but not limited, to A. hydrophila, Psychromonas spp. including, but not limited to, P. ingrahamii, Photobacterium spp. including, but not limited, to P. profundum, Vibrio spp. including, but not limited, to V angustum, V. cholerae, V alginolyticus, V parahaemolyticus, V vulnificus, V fischeri, V splendidus, Shewanella spp. including, but not limited to, S. amazonensis, S. woodyi, S. frigidimarina, S. paeleana, S. baltica, S. denitrificans, Oceanospirillum spp., Xanthomonas spp. including, but not limited to, X oryzae, X campestris, Chromohalobacter spp. including, but not limited, to C. salexigens, Idiomarina spp. including, but not limited, to I. baltica, Pseudoalteromonas spp. including, but not limited to, P. atlantica, Alteromonas spp., Saccharophagus spp. including, but not limited to, S. degradans, S. marine gamma proteobacterium, S. alpha proteobacterium, Pseudomonas spp. including, but not limited to, P. aeruginosa, P. putida, P. fluorescens, Burkholderia spp. including, but not limited to, B. phytofirmans, B. cenocepacia, B. cepacia, B. ambifaria, B. vietnamensis, B. multivorans, B. dolosa, Methylbacillus spp. including, but not limited to, M. flageliatus, Stenotrophomonas spp. including, but not limited to, S. maltophilia, Congregibacter spp. including, but not limited to, C. litoralis, Serratia spp. including, but not limited to, S. proteamaculans, Marinomonas spp., Xytella spp. including, but not limited to, X fastidiosa, Reinekea spp., Colweffia spp. including, but not limited to, C. psychrerythraea, Yersinia spp. including, but not limited to, Y. pestis, Y. pseudotuberculosis, Methylobacillus spp. including, but not limited to, M flagellatus, Cytophaga spp. including, but not limited to, C. hutchinsonii, Flavobacterium spp. including, but not limited to, F. johnsoniae, Microscilla spp. including, but not limited to, M marina, Polaribacter spp. including, but not limited to, P. irgensii, Clostridium spp. including, but not limited to, C. acetobutylicum, C. beijerenckii, C. cellulolyticum, Coxiella spp. including, but not limited to, C. burnetii. In a further embodiment, the ter is derived from a Treponema denticola or F. succinogenes. In yet another embodiment, the ter is a mutant ter comprising an M11K substitution.

In another embodiment, microorganisms are described that are capable of metabolizing a carbon source for producing n-butanol at a yield of at least 4% of theoretical, and, in some cases, a yield cf over 50% of theoretical. As used herein, the term “yield” refers to the molar yield. For example, the yield equals 100% when one mole of glucose is converted to one mole of n-butanol. In particular, the term “yield” is defined as the mole of product obtained per mole of carbon source monomer and may be expressed as percent. Unless otherwise noted, yield is expressed as a percentage of the theoretical yield. “Theoretical yield” is defined as the maximum moles of product that can be generated per a given mole of substrate as dictated by the stoichiometry of the metabolic pathway used to make the product. For example, the theoretical yield for one typical conversion of glucose to n-butanol is 100%. As such, a yield of n-butanol from glucose of 95% would be expressed as 95% of theoretical or 95% theoretical yield. In one embodiment, the yield is at least 4%, 5%, 6%, 7%, 8%, 9%, 10%, 11% or more. For example, the yield of a recombinant E. coli of the disclosure can generate a yield of 4-15% (e.g., 4%, 5%, 6%, 7%, 8%, 9%, 10%, 11%). In another example, the yield of a recombinant yeast cell can be from 5% to 50%.

In another embodiment, a culture comprises a population microorganism that is substantially homogenous (e.g., from about 70-100% homogenous). In another embodiment, a culture can comprise a combination of micoorganism each having distinct biosynthetic pathways that produced metabolites that can be used by at least one other microorganism in culture leading to the production of n-butanol.

The disclosure provides accession numbers for various genes, homologs and variants useful in the generation of recombinant microorganism described herein. It is to be understood that homologs and variants described herein are exemplary and non-limiting. Additional homologs, variants and sequences are available to those of skill in the art using various databases including, for example, the National Center for Biotechnology Information (NCBI) access to which is available on the World-Wide-Web. Furthermore, the disclosure demonstrates that by reducing oxidation of NADH by competitive pathways, effective n-butanol production and/or coupling NADH utilization more closely to the n-butanol production pathway described herein provides an increase in n-butanol production. Identifying competing (oxidative) pathways in various organism is within the skill in the art and various enzymes in such pathways can be reduced by knocking out the polynucleotide encoding such enzyme or reducing expression. Accordingly, exemplary genes and sequences are provided herein, however, one will recognize the ability to identify homologs in various species as well as enzymes having similar synthetic or catabolic activity based on the teachings herein.

Trans-2-enoyl-CoA reductase is encoded in T. denticola F.succinogens, T. vincentii or F. johnsoniae ter gene. In T. denticoloa TER has the accession number Q73Q47 (see also FIG. 24). In one embodiment the F. succinogens TER comprises the sequence set forth in FIG. 24 and has a MetllLys mutation. Other TER polypeptides are set forth in FIG. 24. In addition, TER proteins can also be identified by generally well known bioinformatics methods, such as BLAST. Examples of TER proteins include, but are not limited to, TERs from species such as: Euglena spp. including, but not limited to, E. gracilis, Aeromonas spp. including, but not limited, to A. hydrophila, Psychromonas spp. including, but not limited to, P. ingrahamii, Photobacterium spp. including, but not limited, to P. profundum, Vibrio spp. including, but not limited, to V. angustum, V. cholerae, V. alginolyticus, V. parahaemolyticus, V. vulnificus, V. fischeri, V. splendidus, Shewanella spp. including, but not limited to, S. amazonensis, S. woodyi, S. frigidimarina, S. paeleana, S. baltica, S. denitrificans, Oceanospirillum spp., Xanthomonas spp. including, but not limited to, X. oryzae, X. campestris, Chromohalobacter spp. including, but not limited, to C. salexigens, Idiomarina spp. including, but not limited, to I. baltica, Pseudoalteromonas spp. including, but not limited to, P. atlantica, Alteromonas spp., Saccharophagus spp. including, but not limited to, S. degradans, S. marine gamma proteobacterium, S. alpha proteobacterium, Pseudomonas spp. including, but not limited to, P. aeruginosa, P. putida, P. fluorescens, Burkholderia spp. including, but not limited to, B. phytofirmans, B. cenocepacia, B. cepacia, B. arnbifaria, B. vietnamensis, B. multivorans, B. dolosa, Methylbacillus spp. including, but not limited to, M. flageliatus, Stenotrophomonas spp. including, but not limited to, S. maltophilia, Congregibacter spp. including, but not limited to, C. litoralis, Serratia spp. including, but not limited to, S. proteamaculans, Marinomonas spp., Xytella spp. including, but not limited to, X. fastidiosa, Reinekea spp., Coiweffia spp. including, but not limited to, C. psychrerythraea, Yersinia spp. including, but not limited to, Y. pestis, Y. pseudotuberculosis, Methylobacillus spp. including, but not limited to, M. flagellatus, Cytophaga spp. including, but not limited to, C. hutchinsonii, Flavobacterium spp. including, but not limited to, F. johnsoniae, Microscilla spp. including, but not limited to, M. marina, Pclaribacter spp. including, but not limited to, P. irgensii, Clostridium spp. including, but not limited to, C. acetobutylicum, C. beijerenckii, C. cellulolyticum, Coxiella spp. including, but not limited to, C. burnetii. In a further embodiment, the Ter is derived from a Treponema denticola or F. succinogenes. In yet another embodiment, the ter is a mutant ter comprising an M11K substitution.

In yet another embodiment, the microorganism comprises expression or over expression or one or more or all of the following AccABCD, npHT7, phaB, PhaJ, Ter, Ccr, Bldh, and/or yqhD. In yet other embodiments, the microorganism comprises one or more knockouts selected from the group consisting of frdBc, idhA, adhE and pta.

The disclosure identifies genes useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutation and silent mutations. Such modified or mutated polynucleotides and polypeptides can be screened for expression of a functional enzyme activity using methods known in the art.

Due to the inherent degeneracy of the genetic code, other polynucleotides which encode substantially the same or a functionally equivalent polypeptide can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

Those of skill in the art will recognize that, due to the degenerate nature of the genetic code, a variety of DNA compounds differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as they modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for generating metabolites are encompassed by the microorganisms and methods provided herein. The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which can also be referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A typical algorithm used comparing a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences can be determined using FASTA with its default parameters (a word size of 2 and the PAM250 scoring matrix), as provided in GCG Version 6.1, hereby incorporated herein by reference.

It is understood that a range of microorganisms can be modified to include a recombinant metabolic pathway suitable for the production of e.g., 1-propanol, isobutanol, 1-butanol, 2-methyl 1-butanol, 3-methyl 1-butanol or 2-phenylethanol. It is also understood that various microorganisms can act as “sources” for genetic material encoding target enzymes suitable for use in a recombinant microorganism provided herein. The term “microorganism” includes prokaryotic and eukaryotic photsynthetic microbial species. The terms “microbial cells” and “microbes” are used interchangeably with the term microorganism.

“Bacteria”, or “eubacteria”, refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

Photoautotrophic bacteria are typically Gram-negative rods which obtain their energy from sunlight through the processes of photosynthesis. In this process, sunlight energy is used in the synthesis of carbohydrates, which in recombinant photoautotrophs can be further used as intermediates in the synthesis of biofuels. In other embodiment, the photoautotrophs serve as a sournce of carbohydrates for use by non-photosynthetic microorganism (e.g., recombinant E.coli) to produce biofuels by a metabolically engineered microorganism. Certain photoautotrophs called anoxygenic photoautotrophs grow only under anaerobic conditions and neither use water as a source of hydrogen nor produce oxygen from photosynthesis. Other photoautotrophic bacteria are oxygenic photoautotrophs. These bacteria are typically cyanobacteria. They use chlorophyll pigments and photosynthesis in photosynthetic processes resembling those in algae and complex plants. During the process, they use water as a source of hydrogen and produce oxygen as a product of photosynthesis.

Cyanobacteria include various types of bacterial rods and cocci, as well as certain filamentous forms. The cells contain thylakoids, which are cytoplasmic, platelike membranes containing chlorophyll. The organisms produce heterocysts, which are specialized cells believed to function in the fixation of nitrogen compounds.

The term “recombinant microorganism” and “recombinant host cell” are used interchangeably herein and refer to microorganisms that have been genetically modified to express or over-express endogenous nucleic acid sequences, or to express non-endogenous sequences, such as those included in a vector. The nucleic acid sequence generally encodes a target enzyme involved in a metabolic pathway for producing a desired metabolite as described above. Accordingly, recombinant microorganisms described herein have been genetically engineered to express or over-express target enzymes not previously expressed or over-expressed by a parental microorganism. It is understood that the terms “recombinant microorganism” and “recombinant host cell” refer not only to the particular recombinant microorganism but to the progeny or potential progeny of such a microorganism.

A “parental microorganism” refers to a cell used to generate a recombinant microorganism. The term “parental microorganism” describes a cell that occurs in nature, i.e. a “wild-type” cell that has not been genetically modified. The term “parental microorganism” also describes a cell that has been genetically modified but which does not express or over-express a target enzyme e.g., an enzyme involved in the biosynthetic pathway for the production of a desired metabolite such as 1-propanol, isobutanol, 1-butanol, 2-methyl 1-butanol, 3-methyl 1-butanol or 2-phenylethanol. For example, a wild-type microorganism can be genetically modified to express or over express a first target enzyme such as thiolase. This microorganism can act as a parental microorganism in the generation of a microorganism modified to express or over-express a second target enzyme e.g., hydroxybutyryl CoA dehydrogenase. In turn, the microorganism modified to express or over express e.g., thiolase and hydroxybutyryl CoA dehydrogenase can be modified to express or over express a third target enzyme e.g., crotonase. Accordingly, a parental microorganism functions as a reference cell for successive genetic modification events. Each modification event can be accomplished by introducing a nucleic acid molecule in to the reference cell. The introduction facilitates the expression or over-expression of a target enzyme. It is understood that the term “facilitates” encompasses the activation of endogenous nucleic acid sequences encoding a target enzyme through genetic modification of e.g., a promoter sequence in a parental microorganism. It is further understood that the term “facilitates” encompasses the introduction of exogenous nucleic acid sequences encoding a target enzyme in to a parental microorganism.

In another embodiment a method of producing a recombinant microorganism that converts a suitable carbon substrate to e.g., 1-propanol, isobutanol, 1-butanol, 2-methyl 1-butanol, 3-methyl 1-butanol or 2-phenylethanol is provided. The method includes transforming a microorganism with one or more recombinant nucleic acid sequences as described above and elsewhere herein. Nucleic acid sequences that encode enzymes useful for generating metabolites including homologs, variants, fragments, related fusion proteins, or functional equivalents thereof, are used in recombinant nucleic acid molecules that direct the expression of such polypeptides in appropriate host cells, such as bacterial or yeast cells. It is understood that the addition of sequences which do not alter the encoded activity of a nucleic acid molecule, such as the addition of a non-functional or non-coding sequence, is a conservative variation of the basic nucleic acid. The “activity” of an enzyme is a measure of its ability to catalyze a reaction resulting in a metabolite, i.e., to “function”, and may be expressed as the rate at which the metabolite of the reaction is produced. For example, enzyme activity can be represented as the amount of metabolite produced per unit of time or per unit of enzyme (e.g., concentration or weight), or in terms of affinity or dissociation constants.

A “protein” or “polypeptide”, which terms are used interchangeably herein, comprise's one or more chains of chemical building blocks called amino acids that are linked together by chemical bonds called peptide bonds. An “enzyme” means any substance, composed wholly or largely of protein, that catalyzes or promotes, more or less specifically, one or more chemical or biochemical reactions. The term “enzyme” can also refer to a catalytic polynucleotide (e.g., RNA or DNA). A “native” or “wild-type” protein, enzyme, polynucleotide, gene, or cell, means a protein, enzyme, polynucleotide, gene, or cell that occurs in nature.

It is understood that the nucleic acid sequences described above include “genes” and that the nucleic acid molecules described above include “vectors” or “plasmids.” For example, a nucleic acid sequence encoding a keto thiolase can be encoded by an atoB gene or homolog thereof, or an fadA gene or homolog thereof. Accordingly, the term “gene”, also called a “structural gene” refers to a nucleic acid sequence that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence. The term “nucleic acid” or “recombinant nucleic acid” refers to polynucleotides such as deoxyribonucleic acid (DNA), and, where appropriate, ribonucleic acid (RNA). The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence.

The term “operon” refers two or more genes which are transcribed as a single transcriptional unit from a common promoter. In some embodiments, the genes comprising the operon are contiguous genes. It is understood that transcription of an entire operon can be modified (i.e., increased, decreased, or eliminated) by modifying the common promoter. Alternatively, any gene or combination of genes in an operon can be modified to alter the function or activity of the encoded polypeptide. The modification can result in an increase in the activity of the encoded polypeptide. Further, the modification can impart new activities on the encoded polypeptide. Exemplary new activities include the use of alternative substrates and/or the ability to function in alternative environmental conditions.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

“Transformation” refers to the process by which a vector is introduced into a host cell. Transformation (or transduction, or transfection), can be achieved by any one of a number of means including electroporation, microinjection, biolistics (or particle bombardment-mediated delivery), or agrobacterium mediated transformation.

Those of skill in the art will recognize that, due to the degenerate nature of the genetic code, a variety of DNA compounds differing in their nucleotide sequences can be used to encode a given amino acid sequence of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate embodiments of the disclosure.

The disclosure provides nucleic acid molecules in the form of recombinant DNA expression vectors or plasmids, as described in more detail below, that encode one or more target enzymes. Generally, such vectors can either replicate in the cytoplasm of the host microorganism or integrate into the chromosomal DNA of the host microorganism. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) forms.

Provided herein are methods for the heterologous expression of one or more of the biosynthetic genes involved in 1-propanol, isobutanol, 1-butanol, 2-methyl 1-butanol, 3-methyl 1-butanol, and/or 2-phenylethanol biosynthesis and recombinant DNA expression vectors useful in the method. Thus, included within the scope of the disclosure are recombinant expression vectors that include such nucleic acids. The term expression vector refers to a nucleic acid that can be introduced into a host microorganism or cell-free transcription and translation system. An expression vector can be maintained permanently or transiently in a microorganism, whether as part of the chromosomal or other DNA in the microorganism or in any cellular compartment, such as a replicating vector in the cytoplasm. An expression vector also comprises a promoter that drives expression of an RNA, which typically is translated into a polypeptide in the microorganism or cell extract. For efficient translation of RNA into protein, the expression vector also typically contains a ribosome-binding site sequence positioned upstream of the start codon of the coding sequence of the gene to be expressed. Other elements, such as enhancers, secretion signal sequences, transcription termination sequences, and one or more marker genes by which host microorganisms containing the vector can be identified and/or selected, may also be present in an expression vector. Selectable markers, i.e., genes that confer antibiotic resistance or sensitivity, are used and confer a selectable phenotype on transformed cells when the cells are grown in an appropriate selective medium.

The various components of an expression vector can vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those.that function in eukaryotic or prokaryotic host microorganisms. Promoters can comprise regulatory sequences that allow for regulation of expression relative to the growth of the host microorganism or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (trp), beta-lactamase (bla), bacteriophage lambda PL, and T5 promoters. In addition, synthetic promoters, such as the tac promoter (U.S. Pat. No. 4,551,433), can also be used. For E. coli expression vectors, it is useful to include an E. coli origin of replication, such as from pUC, p1P, pl, and pBR.

Thus, recombinant expression vectors contain at least one expression system, which, in turn, is composed of at least a portion of PKS and/or other biosynthetic gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

Due to the inherent degeneracy of the genetic code, other nucleic acid sequences which encode substantially the same or a functionally equivalent amino acid sequence can also be used to clone and express the polynucleotides encoding such enzymes. As previously noted, the term “host cell” is used interchangeably with the term “recombinant microorganism” and includes any cell type which is suitable for producing e.g., 1-propanol, isobutanol, 1-butanol, 2-methyl 1-butanol, 3-methyl 1-butanol and/or 2-phenylethanol and susceptible to transformation with a nucleic acid construct such as a vector or plasmid.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

A nucleic acid of the disclosure can be amplified using cDNA, mRNA or alternatively, genomic DNA, as a template and appropriate oligonucleotide primers according to standard PCR amplification techniques and those procedures described in the Examples section below. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

It is also understood that an isolated nucleic acid molecule encoding a polypeptide homologous to the enzymes described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the nucleic acid sequence by standard techniques, such as site-directed mutagenesis and PCR-mediated mutagenesis. In contrast to those positions where it may be desirable to make a non-conservative amino acid substitutions (see above), in some positions it is preferable to make conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In another embodiment a method for producing e.g., 1-propanol, isobutanol, 1-butanol, 2-methyl 1-butanol, 3-methyl 1-butanol or 2-phenylethanol is provided. The method includes culturing a recombinant photoautotroph microorganism(s) or culture comprising a photoautotroph and a recombinant non-photosynthetic or photoheterotroph microorganism as provided herein in the presence of a suitable substrate (e.g., CO₂) and under conditions suitable for the conversion of the substrate to 1-propanol, isobutanol, 1-butanol, 2-methyl 1-butanol, 3-methyl 1-butanol or 2-phenylethanol. The alcohol produced by a microorganism or culture provided herein can be detected by any method known to the skilled artisan. Culture conditions suitable for the growth and maintenance of a recombinant microorganism provided herein are described in the Examples below. The skilled artisan will recognize that such conditions can be modified to accommodate the requirements of each microorganism.

As previously discussed, general texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel at al., eds., Current Protocols, a joint venture between Greene Publishing Associates, Inc. and John Wiley & Sons, Inc., (supplemented through 1999) (“Ausubel”). Examples of protocols sufficient to direct persons of skill through in vitro amplification methods, including the polymerase chain reaction (PCR), the ligase chain reaction (LCR), Qβ-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli at al. (1990) Proc. Nat'l. Acad. Sci. USA 87: 1874; Lomell at al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13: 563-564. Improved methods for cloning in vitro amplified nucleic acids are described in Wallace et al., U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

The disclosure provides accession numbers for various genes, homologs and variants useful in the generation of recombinant microorganism described herein. It is to be understood that homologs and variants described herein are exemplary and non-limiting. Additional homologs, variants and sequences are available to those of skill in the art using various databases including, for example, the National Center for Biotechnology Information (NCBI) access to which is available on the World-Wide-Web.

3 hydroxy-butyryl-coA-dehydrogenase catalyzes the conversion of acetoacetyl-coA to 3-hydroxybutyryl-CoA. Depending upon the organism used a heterologous 3-hydroxy-butyryl-coA-dehydrogenase can be engineered for expression in the organism. Alternatlively a native 3-hydroxy-butyryl-coA-dehydrogenase can be overexpressed. 3-hydroxy-butyryl-coA-dehydrogenase is encoded in C.acetobuylicum by hbd. HBD homologs and variants are known. For examples, such homologs and variants include, for example, 3-hydroxybutyryl-CoA dehydrogenase (Clostridium acetobutylicum ATCC 824) gi|15895965|refINP349314.11(15895965); 3-hydroxybutyryl-CoA dehydrogenase (Bordetella pertussis Tohama I) gi|33571103|embICAE40597.11(33571103); 3-hydroxybutyryl-CoA dehydrogenase (Streptomyces coelicolor A3(2)) gi|21223745|refINP_{—}629524.11(21223745); 3-hydroxybutyryl-CoA dehydrogenase gi|1055222|gbIAAA95971.11(1055222); 3-hydroxybutyryl-CoA dehydrogenase (Clostridium perfringens str. 13) gi|18311280|refINP_{—}563214.11(18311280); 3-hydroxybutyryl-CoA dehydrogenase (Clostridium perfringens str. 13) gi|18145963|dbj1BAB82004.11(18145963) each sequence associated with the accession number is incorporated herein by reference in its entirety.

Crotonase catalyzes the conversion of 3-hydroxybutyryl-CoA to crotonyl-CoA. Depending upon the organism used a heterologous Crotonase can be engineered for expression in the organism. Alternatlively a native Crotonase can be overexpressed. Crotonase is encoded in C.acetobuylicum by crt. CRT homologs and variants are known. For examples, such homologs and variants include, for example, crotonase (butyrate-producing bacterium L2-50) gii|19370267|gb|ABL68062.1| (119370267); crotonase gi|1055218|gb|AAA95967.1| (1055218); crotonase (Clostridium perfringens NCTC 8239) gi|168218170|ref|ZP_{—}02643795.1| (168218170); crotonase (Clostridium perfringens CPE str. F4969) gi|168215036|ref|ZP_{—}02640661.1| (168215036); crotonase (Clostridium perfringens E str. JGS1987) gi|168207716|ref|ZP_{—}02633721.1| (168207716); crotonase (Azoarcus sp. EbN1) gi|56476648|ref|YP158237.1| (56476648); crotonase (Roseovarius sp. TM1035) gi|149203066|ref|ZP_{—}01880037.1| (149203066); crotonase (Roseovarius sp. TM1035) gi|149143612|gb|EDM31648.1| (149143612); crotonase; 3-hydroxbutyryl-CoA dehydratase (Mesorhizobium loti MAFF303099) gi|14027492|dbj|BAB53761.1| (14027492); crotonase (Roseobacter sp. SK209-2-6) gi|126738922|ref|ZP_{—}01754618.1| (126738922); crotonase (Roseobacter sp. SK209-2-6) gi|126720103|gb|EBA16810.1| (126720103); crotonase (Marinobacter sp. ELB17) gi|126665001|ref|ZP_{—}01735984.1| (126665001); crotonase (Marinobacter sp. ELB17) gi|126630371|gb|EBA00986.1| (126630371); crotonase (Azoarcus sp. EbN1) gi|56312691|emb|CAI07336.1| (56312691); crotonase (Marinomonas sp. MED121) gi|86166463|gb|EAQ67729.1| (86166463); crotonase (Marinomonas sp. MED121) gi|87118829|ref|ZP_{—}01074728.1| (87118829); crotonase (Roseovarius sp. 217) gi|85705898|ref|ZP_{—}01036994.1| (85705898); crotonase (Roseovarius sp. 217) gi|85669486|gb|EAQ24351.1| (85669486); crotonase gi|1055218|gb|AAA95967.1| (1055218); 3-hydroxybutyryl-CoA dehydratase (Crotonase) gi|1706153|sp|P52046.1|CRT_CLOAB(1706153); Crotonase (3-hydroxybutyryl-COA dehydratase) (Clostridium acetobutylicum ATCC 824) gi|15025745|gb|AAK80658.1|AE007768_{—}12 (15025745) each sequence associated with the accession number is incorporated herein by reference in its entirety.

Aldehyde/alcohol dehydrogenase catalyzes the conversion of butyryl-CoA to butyraldehyde and butyraldehyde to 1-butanol. In one embodiment, the aldehyde/alcohol dehydrogenase preferentially catalyzes the conversion of butyryl-CoA to butyraldehyde and butyraldehyde to 1-butanol. Depending upon the organism used a heterologous aldehyde/alcohol dehydrogenase can be engineered for expression in.the organism. Alternatively, a native aldehyde/alcohol dehydrogenase can be overexpressed. aldehyde/alcohol dehydrogenase is encoded in C.acetobuylicum by adhE (e.g., an adhE2). ADHE (e.g., ADHE2) homologs and variants are known. For examples, such homologs and variants include, for example, aldehyde-alcohol dehydrogenase (Clostridium acetobutylicum) gi|3790107|gb|AAD04638.1| (3790107); aldehyde-alcohol dehydrogenase (Clostridium botulinum A str. ATCC 3502) gi|148378348|ref|YP_{—}001252889.1| (148378348); Aldehyde-alcohol dehydrogenase (Includes: Alcohol dehydrogenase (ADH) Acetaldehyde dehydrogenase (acetylating) (ACDH) gi|19858620|sp|P33744.3|ADHE_CLOAB(19858620); Aldehyde dehydrogenase (NAD+) (Clostridium acetobutylicum ATCC 824) gi|15004865|ref|NP_{—}149325.1| (15004865); alcohol dehydrogenase E (Clostridium acetobutylicum) gi|298083|emb|CAA51344.1| (298083); Aldehyde dehydrogenase (NAD+) (Clostridium acetobutylicum ATCC 824) gi|14994477|gb|AAK76907.1|AE001438_{—}160(14994477); aldehyde/alcohol dehydrogenase (Clostridium acetobutylicum) gi|12958626|gb|AAK09379.1|AF321779_{—}1(12958626); Aldehyde-alcohol dehydrogenase, ADHE1 (Clostridium acetobutylicum ATCC 824) gi|15004739|ref|NP_{—}149199.1| (15004739); Aldehyde-alcohol dehydrogenase, ADHE1 (Clostridium acetobutylicum ATCC 824) gi|14994351|gb|AAK76781.1|AE001438_{—}34(14994351); aldehyde-alcohol dehydrogenase E (Clostridium perfringens str. 13) gi|18311513|ref|NP_{—}563447.1| (18311513); aldehyde-alcohol dehydrogenase E (Clostridium perfringens str. 13) gi|18146197|dbj|BAB82237.1| (18146197), each sequence associated with the accession number is incorporated herein by reference in its entirety.

Crotonyl-coA reductase catalyzes the reduction of crotonyl-CoA to butyryl-CoA. Depending upon the organism used a heterologous Crotonyl-coA reductase can be engineered for expression in the organism. Alternatively, a native Crotonyl-coA reductase can be overexpressed. Crotonyl-coA reductase is encoded in S.coelicolor by ccr. CCR homologs and variants are known. For examples, such homologs and variants include, for example, crotonyl CoA reductase (Streptomyces coelicolor A3(2)) gi|21224777|ref|NP_{—}630556.1| (21224777); crotonyl CoA reductase (Streptomyces coelicolor A3(2)) gi|4154068|emb|CAA22721.1| (4154068); crotonyl-CoA reductase (Methylobacterium sp. 4-46) gi|168192678|gb|ACA14625.1| (168192678); crotonyl-CoA reductase (Dinoroseobacter shibae DFL 12) gi|159045393|ref|YP_{—}001534187.1| (159045393); crotonyl-CoA reductase (Salinispora arenicola CNS-205) gi|159039522|ref|YP_{—}001538775.1| (159039522); crotonyl-CoA reductase (Methylobacterium extorquens PA1) gi|163849740|ref|YP_{—}001637783.1| (163849740); crotonyl-CoA reductase (Methylobacterium extorquens PA1) gi|163661345|gb|ABY28712.1| (163661345); crotonyl-CoA reductase (Burkholderia ainbifaria AMMD) gi|115360962|ref|YP_{—}778099.1| (115360962); crotonyl-CoA reductase (Parvibaculum lavamentivorans DS-1) gi|154252073|ref|YP_{—}001412897.1| (154252073); Crotonyl-CoA reductase (Silicibacter sp. TM1040) gi|99078082|ref|YP_{—}611340.1| (99078082); crotonyl-CoA reductase (Xanthobacter autotrophicus Py2) gi|154245143|ref|YP_{—}001416101.1| (154245143); crotonyl-CoA reductase (Nocardioides sp. JS614) gi|119716029|ref|YP_{—}922994.11(119716029); crotonyl-CoA reductase (Nocardioides sp. JS614) gi|119536690|gb|ABL81307.1| (119536690); crotonyl-CoA reductase (Salinispora arenicola CNS-205) gi|157918357|gb|ABV99784.1| (157918357); crotonyl-CoA reductase (Dinoroseobacter shibae DFL 12) gi|157913153|gb|ABV94586.1| (157913153); crotonyl-CoA reductase (Burkholderia ambifaria AMMD) gi|115286290|gb|AB191765.1| (115286290); crotonyl-CoA reductase (Xanthobacter aucotrophicus Py2) gi|154159228|gb|ABS66444.1| (154159228); crotonyl-CoA reductase (Parvibaculum lavamentivorans DS-1) gi|154156023|gb|ABS63240.1| (154156023); crotonyl-CoA reductase (Methylobacterium radiotolerans JCM 2831) gi|170654059|gb|ACB23114.1| (170654059); crotonyl-CoA reductase (Burkholderia graminis C4D1M) gi|170140183|gb|EDT08361.1| (170140183); crotonyl-CoA reductase (Methylobacterium sp. 4-46) gi|168198006|gb|ACA19953.1| (168198006); crotonyl-CoA reductase (Frankia sp. EANlpec) gi|158315836|ref|YP_{—}001508344.1| (158315836), each sequence associated with the accession number is incorporated herein by reference in its entirety.

### EXAMPLES

### Materials and Methods

Chemicals and reagents. All chemicals were purchased from Sigma-Aldrich (St. Louis, Mo.) or Fisher Scientifics (Pittsburgh, Pa.) unless otherwise specified. iProof high-fidelity DNA polymerase was purchased from Bio-Rad (Hercules, Calif.). Restriction enzymes, Phusion DNA polymerase, and ligases were purchased from New England Biolabs (Ipswich, Mass.). T5-Exonuclease was purchased from Epicentre Biotechnologies (Madison, Wis.). KOD and KOD xtreme DNA polymerases were purchased from EMD biosciences (Gibbstown, N.J.).

Culture medium and condition. All S. elongatus 7942 strains were grown on modified BG-11 (1.5 g/L NaNO₃, 0.0272 g/L CaCl₂.2H₂O, 0.012 g/L ferric ammonium citrate, 0.001 g/L Na₂EDTA, 0.040 g/L K₂HPO₄, 0.0361 g/L MgSO₄.7H₂O, 0.020 g/L Na₂CO₃, 1000× trace mineral (1.43 g H₃BO₃, 0.905 g/L MnCl₂.4H₂O, 0.111 g/L ZnSO₄.7H₂O, 0.195 g/L Na₂MoO₄.2H₂O, 0.0395 g CuSO₄.5H₂O, 0.0245 g Co(NO₃)2.6H₂O), 0.00882 g/L sodium citrate dihydrate) agar (1.5% w/v) plates. All S. elongatus 7942 strains were cultured in BG-11 medium containing 50 mM NaHCO₃ in 250 mL screw-capped flasks. Cultures were grown under 100 μE/s/m² light condition at 30° C. Cell growth was monitored by measuring OD₇₃₀ with Beckman Coulter DU800 spectrophotometer.

DNA manipulations. All chromosomal manipulations were carried out by recombination of plasmid DNA into S. elongatus 7942 genome at neutral site I (NSI) and II (NSII). All plasmid were constructed using the isothermal DNA assembly method. Plasmids were constructed in E. coli XL-1 strain for propagation and storage (SI Table 1).

SI TABLE 1
Strain and plasmid list

| | | |
|---|---|---|
| Strain | Relevant genotypes | Reference |

Cyanobacteria Strains

| | | |
|---|---|---|
| PCC 7942 | Wild-type Synechococcus elongatus PCC 7942 | S. S. Golden |
| EL9 | His-tagged T. denticola ter integrated at NSI in PCC7942 genome | (17) |
| EL14 | His-tagged T. denticola ter integrated at NSI and atoB, adhE2, crt, hbd | (17) |
| | integrated at NSII in PCC7942 genome | |
| EL18 | His-tagged T. denticola ter integrated at NSI and atoB, bldh, yqhD, crt, hbd | This work |
| | integrated at NSII in PCC7942 genome | |
| EL20 | His-tagged T. denticola ter integrated at NSI and nphT7, adhE2, crt, hbd | This work |
| | integrated at NSII in PCC7942 genome | |
| EL21 | His-tagged T. denticola ter integrated at NSI and nphT7, bldh, yqhD, crt, hbd | This work |
| | integrated at NSII in PCC7942 genome | |
| EL22 | His-tagged T. denticola ter integrated at NSI and nphT7, bldh, yqhD, phaJ, | This work |
| | phaB integrated at NSII in PCC7942 genome | |
| EL23 | His-tagged T. denticola ter integrated at NSI and atoB, bldh, yqhD, phaJ, phaB | This work |
| | integrated at NSII in PCC7942 genome | |
| EL24 | His-tagged T. denticola ter integrated at NSI and nphT7, adhE2, phaJ, phaB | This work |
| | integrated at NSII in PCC7942 genome | |

E. coli strains

| BW25113 | rrnB_{T14} ΔlacZ_{WJ16} hsdR514 ΔaraBAD_{AH33} ΔrhaBAD_{LD78} | (54) |
|---|---|---|
| XL-1 blue | recA1 endA1 gyrA96 thi-1 hsdR17 supE44 relA1 lac [F′ proAB lacl^{q}ZΔM15 Tn10 | Stratagene |
| | (Tet^{R})] | |
| JCL299 | BW25113 ΔldhA ΔadhE ΔfrdBC Δpta/F′ [traD36, proAB+, lacl^{q} ZΔM15 (Tet^{R})] | (16) |
| Plasmid | genotypes | Reference |
| pCDFDuet | Spec^{R}; CDF ori; pT7::MCS | Novagen |
| pCDF-nphT7 | Spec^{R}; CDF ori; pT7::nphT7 (his tagged) | This work |
| pCDF-atoB | Spec^{R}; CDF ori; pT7::atoB (his tagged) | This work |
| pCS27 | Kan^{R}; P15A ori; pLlacO1::MCS | (1) |
| pDK26 | Amp^{R}; ColE1 ori; pLlacO1::bktB.adhE2.crt.paaH1 | Yasumasa |
| | | Dekishima |
| pEL11 | Amp^{R}; ColE1 ori; pLlacO1::atoB.adhE2.crt.hbd | (1) |
| pEL29 | Kan^{R}; pUC ori; ccr-phaJ-phaB | This work |
| pEL37 | Kan^{R}; NSII targeting; pLlacO1::atoB.adhE2.crt.hbd | (17) |
| pEL52 | Amp^{R}; pUC ori; PT5::nphT7 | This work |
| pEL53 | Amp^{R}; ColE1 ori; pLlacO1::nphT7.adhE2.crt.hbd | This work |
| pEL54 | Amp^{R}; ColE1 ori; pLlacO1::ato8.bldh.yqhD.crt.hbd | This work |
| pEL56 | Kan^{R}; NSII targeting; pLlacO1::nphT7.adhE2.crt.hbd | This work |
| pEL57 | Kan^{R}; NSII targeting; pLlacO1::atoB.bldh.yqhD.crt.hbd | This work |
| pEL59 | Kan^{R}; NSII targeting; pLlacO1::nphT7.bldh.yqhD.crt.hbd | This work |
| pEL70 | Kan^{R}; NSII targeting; pLlacO1::nphT7.bldh.yqhD.phaJ.phaB | This work |
| pEL71 | Kan^{R}; NSII targeting; pLlacO1::atoB.bldh.yqhD.phaJ.phaB | This work |
| pEL73 | Kan^{R}; NSII targeting; pLlacO1::nphT7.adhE2.phaJ.phaB | This work |
| pEL75 | Amp^{R}; ColE1 ori; pLlacO1::bktB.bldh.yqhD.crt.paaH1 | This work |
| pEL76 | Amp^{R}; ColE1 ori; pLlacO1::bktB.aldh(CK).yqhD.crt.paaH1 | This work |
| pEL77 | Amp^{R}; ColE1 ori; pLlacO1::bktB.aldh(GT).yqhD.crt.paaH1 | This work |
| pEL78 | Amp^{R}; ColE1 ori; pLlacO1::bktB.eutE.yqhD.crt.paaH1 | This work |
| pEL79 | Amp^{R}; ColE1 ori; pLlacO1::bktB.aldh(CB).crt.paaH1 | This work |
| pEL80 | Amp^{R}; ColE1 ori; pLlacO1::bktB.aldh(BAA117).yqhD.crt.paaH1 | This work |
| pEL90 | Kan^{R}; P15A ori; pLlacO1::bamb6224 (his-tagged) | This work |
| pEL91 | Kan^{R}; P15A ori; pLlacO1::gox0115 (his-tagged) | This work |
| pEL92 | Kan^{R}; P15A ori; pLlacO1;:hp0202 (his-tagged) | This work |
| pEL93 | Kan^{R}; P15A ori; pLlacO1::lmo2202 (his-tagged) | This work |
| pEL94 | Kan^{R}; P15A ori; pLlacO1::pae-fabH2 (his-tagged) | This work |
| pEL95 | Kan^{R}; P15A ori; pLlacO1::sav-fabH4 (his-tagged) | This work |
| pEL96 | Kan^{R}; P15A ori; pLlacO1::sco5888 (his-tagged) | This work |

Kan^{R}, kanamycin resistance;
Amp^{R}, ampicillin resistance.
atoB (E. coli), thiolase;
nphT7 (Streptomyces sp. strain CL190), acetoacetyl-CoA synthase;
phaB (R. Eutropha), acetoacetyl-CoA reductase;
phaJ (A. caviae), (R)-specific enoyl-CoA hydratase;
hbd (C. acetobutylicum), 3-hydroxybutyryl-CoA dehydrogenase;
crt (C. acetobutylicum), crotonase;
ter (T. denticola), Trans-2-enoyl-CoA reductase;
bldh (C. saccharoperbutylacetonicum), butyraldehyde dehydrogenase;
paaH1 (R. eutropha), 3-hydroxybutyryl-CoA dehydrogenase;
yqhD (E. coli), NADP-dependent alcohol dehydrogenase;
adhE2 (C. acetobutylicum), bifunctional alcohol/aldehyde dehydrogenase.
bktb (R. Eutropha), thiolase;
aldh (C. kluyveri, C. beijerinckii, C. saccharobutylicum, or G. thermoglucosidasius), aldehyde dehydrogenase;
eutE (E. coli), aldehyde dehydrogenase;
KASIII like enzymes:
bamb6224 (Burkholderia ambifaria),
gox0115 (Gluconobacter oxydans),
hp0202 (Helicobacter pylori),
lmo2202 (Listeria monocytogenes),
pae-fabH2 (Pseudomonas aeruginosa),
sav-fabH4 (Streptomyces avermitilis),
sco5888 (Streptomyces coelicolor).

Plasmid constructions. The plasmids used and constructed in this work are listed in SI Table 1 and briefly described below. The sequences of primers used are listed in Table 2. Plasmid pEL29 was synthesized by Genewiz Inc. Plasmid pEL52 was synthesized by DNA 2.0.

Plasmid pEL53 was constructed by assembling a nphT7 fragment and a pEL11 without atoB fragment. nphT7 fragment was amplified by PCR with primers rEL-335 and rEL-336 with pEL52 as template. pEL11 without atoB fragment was amplified by PCR with primers rEL-333 and rEL-334 with pEL11 as template.

Plasmid pEL54 was constructed by assembling a bldh fragment, a yqhD fragment, and a pEL11 without adhE2 fragment. bldh fragment was amplified by PCR with primers rEL-329 and rEL-330 with Clostridium saccharoperbutylacetonicum NI-4 genome as template. yqhD fragment was amplified by PCR with primers rEL-331 and rEL-332 with E. coli genome as template. pEL11 without adhE2 fragment was amplified by PCR with primers rEL-327 and rEL-328 with pEL11 as template.

Plasmid pEL56 was constructed by assembling a NSII vector fragment and a pEL53 coding sequence fragment. NSII vector fragment was amplified by PCR with primers rEL-217 and rEL-253 with pEL37 as template. pEL53 coding sequence fragment was amplified by PCR with primers rEL-254 and rEL-255 with pEL53 as template.

Plasmid pEL57 was constructed by assembling a NSII vector fragment and a pEL54 coding sequence fragment. NSII vector fragment was amplified by PCR with primers rEL-217 and rEL-253 with pEL37 as template. pEL54 coding sequence fragment was amplified by PCR with primers rEL-254 and rEL-255 with pEL54 as template.

Plasmid pEL59 was constructed by assembling a NSII vector fragment, a pEL54 coding sequence without atoB fragment, and a nphT7 fragment. NSII vector fragment was amplified by PCR with primers rEL-217 and rEL-253 with pEL37 as template. pEL54 coding sequence without atoB fragment was amplified by PCR with primers rEL-352 and rEL-255 with pEL54 as template. nphT7 fragment was amplified by PCR with primers rEL-254 and rEL-351.

Plasmid pEL70 was constructed by assembling a pEL59 without crt.hbd fragment and a phaJ.phaB fragment. pEL59 without crt.hbd fragment was amplified by PCR with primers rEL-390 and rEL-391 with pEL59 as template. phaJ.phaB fragment was amplified by PCR with primers rEL-392 and rEL-393 with pEL29 as template.

Plasmid pEL71 was constructed by assembling a pEL57 without crt.hbd fragment and a phaJ.phaB fragment. pEL57 without crt.hbd fragment was amplified by PCR with primers rEL-390 and rEL-391 with pEL57 as template. phaJ.phaB fragment was amplified by PCR with primers rEL-392 and rEL-393 with pEL29 as template.

Plasmid pEL73 was constructed by assembling a pEL56 without crt.hbd fragment and a phaJ.phaB fragment. pEL56 without crt.hbd fragment was amplified by PCR with primers rEL-390 and rEL-398 with pEL56 as template. phaJ.phaB fragment was amplified by PCR with primers rEL-399 and rEL-393 with pEL70 as template.

Plasmids pEL75, pEL76, pEL77, pEL78, pEL79, and pEL80 were constructed by assembling a pDK26 without adhE2 fragment and an aldehyde dehydrogenase gene from Clostridium saccharoperbutylacetonicum NI-4, Clostridium Kluyveri, Geobacillus thermoglucosidasius, Escherichia coli, Clostridium beijerinckii NCIMB 8052, and Clostridium saccharobutylicum ATCC BAA-117, respectively. pDK26 without adhE2 fragment was amplified by PCR using primers rEL-403 and rEL-404 with pDK26 as template. C. saccharoperbutylacetonicum NI-4 bldh fragment was amplified by primers rEL-332 and rEL-394 with C. saccharoperbutylacetonicum NI-4 genome as template. C. Kluyveri bldh fragment was amplified by primers rEL-405 and rEL-406 with C. kluyveri genome as template. G. thermoglucosidasius bldh fragment was amplified by primers rEL-407 and rEL-408 with G. thermoglucosidasius genome as template. E. coli EutE fragment was amplified by primers rEL-409 and rEL-410 with E. coli genome as template. C. beijerinckii NCIMB 8052 bldh fragment was amplified by primers rEL-411 and rEL-412 with C. beijerinckii NCIMB 8052 genome as template. C. saccharobutylicum ATCC BAA-117 bldh fragment was amplified by primers rEL-413 and rEL-414 with C. saccharobutylicum ATCC B4A-117 genome as template.

Plasmids pEL90 to pEL96 were constructed by assembling the KASIII-like genes with a vector fragment. Vector fragment was amplified with primers rEL-455 and rEL-456 with pCS27 as the template. bamb6224 was amplified with primers rEL-457 and rEL-458 with Burkholderia ambifaria gDNA as template. gox0115 was amplified with primers rEL-459 and rEL-460 with Gluconobacter oxydans gDNA as template. hp0202 was amplified with primers rEL-461 and rEL-462 with Helicobacter pylori gDNA as template. 1mo2202 was amplified with primers rEL-463 and rEL-464 with Listeria monocytogenes gDNA as template. pae-fabH2 was amplified with primers rEL-467 and rEL-468 with Pseudomonas aeruginosa gDNA as template. sav-fabH4 was amplified with primers rEL-469 and rEL-470 with Streptomyces avermitilis gDNA as template. sco5888 was amplified with primers rEL-471 and rEL-472 with Streptomyces coelicolor gDNA as template.

Strain construction. The strains used and constructed are listed in SI Table 1. Briefly, strain EL18 was constructed by recombination of plasmids pEL57 NSII of Strain EL9 (SI Table 1 for relevant genotypes). Strain EL20 was constructed by recombination of plasmids pEL56 into NSII of strain EL9. Strain EL21 was constructed by recombination of plasmids pEL59 into NSII of strain EL9. Strain EL22 was constructed by recombination of plasmids pEL70 into NSII of strain EL9. Strain EL23 was constructed by recombination of plasmids pEL71 into NSII of strain EL9. Strain EL24 was constructed by recombination of plasmids pEL73 into NSII of strain EL9.

Plasmid transformation. S. elongacus 7942 strains were transformed by incubating cells at mid-log phase (OD₇₃₀ of 0.4 to 0.6) with 2 μg of plasmid DNA overnight in dark. The culture was then spread on BG-11 plates with appropriate antibiotics for selection of successful recombination. For selection and culture maintenance, 20 μg/ml spectinomycin and 10 μg/ml kanamycin were added into BG-11 agar plates and BG-11 medium where appropriate. Colonies grown on BG-11 agar plates were grown in liquid culture. Genomic DNA was then prepared from the liquid culture and analyzed by PCR using gene-specific primers (SI Table 2) to verify integration of inserted genes into the recombinant strain. In all cases, four individual colonies were analyzed and propagated for downstream tests.

TABLE 2
SI Primer Sequences

| Primers | Sequence (5′−>3′) | Used for plasmid |
|---|---|---|
| rEL-333 | TTGCGCTGATCGAGTGGTAAGCATGCAGGAGAAAGGTACCATGAAAG | pEL53 |
| rEL-334 | ATGCGGAAGCGGACGTCGGTCATGGTACCTTTCTCCTCTTTAATGAATTCGGTC | pEL53 |
| rEL-335 | CCGAATTCATTAAAGAGGAGAAAGGTACCATGACCGACGTCCGCTTCCGCATCA | pEL53; nphT7 gene specific |
| rEL-327 | AGGAGATATACCATGGAACTAAACAATGTCATCC | pEL54 |
| rEL-328 | TTAATTCAACCGTTCAATCACCATCGC | pEL54 |
| rEL-329 | GGTTGAATTAAGCATGCAGGAGAAAGGTACCATGATTAAAGACACGCTAGTTTCTATAAC | pEL54 |
| rEL-330 | GTTGTTCATGGTATATCTCCTTTAACCGGCGAGTACACATCTTCTTTGTC | pEL54 |
| rEL-331 | GTACTCGCCGGTTAAAGGAGATATACCATGAACAACTTTAATCTGCACACCCC | pEL54; yqhD specific |
| rEL-332 | TTGTTTAGTTCCATGGTATATCTCCTTCTAGATTAGCGGGCGGCTTCGTATATACGGCGG | pEL54; yqhD specific |
| rEL-217 | CTTTAATGAATTCGGTCAGTGCGTCCT | pEL56, pEL57, pEL59 |
| rEL-153 | ACGCGTGCTAGAGGCATCAAATAAA | pEL56, pEL57, pEL59 |
| rEL-254 | AGGACGCACTGACCGAATTCATTAAAG | pEL56, pEL57, pEL59 |
| rEL-255 | TTTATTTGATGCCTCTAGCACGCGTTTATTTTGAATAATCGTAGAAACCTTTTCCTG | pEL56, pEL57, pEL59 |
| rEL-351 | CATGGTACCTTTCTCCTGCATGCTTACCACTCGATCAGCGCAAAGCTCGC | pEL59 |
| rEL-352 | TAAGCATGCAGGAGAAAGGTACCATGATTAAAGACACGCTAGTTTC | pEL59 |
| rEL-390 | TAAACGCGTGCTAGAGGCATCAAATA | pEL70, pEL71, pEL73 |
| rEL-391 | GCAGACATGGTATATCTCCTTTAGCGGGGCGGCTTCGTATATACGGC | pEL70, pEL71 |
| rEL-392 | ACGAAGCCGCCCGCTAAAGGAGATATACCATGTCTGCGCAATC | pEL70, pEL71 |
| rEL-393 | TTGATGCCTCTAGCACGCGTTTAACCCATGTGCAGACCACCGTTC | pEL70, pEL71, pEL73 |
| rEL-398 | CATGGTATATCTCCTTTAAAATGATTTTATATAGATATCCTTAAGTTCAC | pEL73 |
| rEL-399 | ATATCTATATAAAATCATTTTAAAGGAGATATACCATGTCTGCGC | pEL73 |
| rEL-403 | TAAAGGAGATATACCATGAACAACTTTAATCTGC | pEL75, pEL76, pEL77, |
| | | pEL78, pEL79, pEL80 |
| rEL-404 | CTTTCTCCTGCATGCTTAGATACGC | pEL75, pEL76, pEL77, |
| | | pEL78, pEL79, pEL80 |
| rEL-332 | TTGTTTAGTTCCATGGTATATCTCCTTCTAGATTAGCGGGCGGCTTCGTATATACGGCGG | pEL75 |
| rEL-394 | ATGCAGGAGAAAGGTACCATGATTAAAGACACGCTAGTTTCTATAAC | pEL75 |
| rEL-405 | AGCGTATCTAAGCATGCAGGAGAAAGGTACCATGGAGATAATGGATAAGGACTTACAGTC | pEL76 |
| rEL-406 | TAAAGTTGTTCATGGTATATCTCCTTTAAAGATTTAATTTAGCCATTATATGCTTTTAC | pEL76 |
| rEL-407 | GTATCTAAGCATGCAGGAGAAAGGTACCATGGATGCACAAAAAATTGAGAAACTTG | pEL77 |
| rEL-408 | AGTTGTTCATGGTATATCTCCTTTATCTTATCGACAAAGCATCCACTAGG | pEL77 |
| rEL-409 | CGTATCTAAGCATGCAGGAGAAAGGTACCATGAATCAACAGGATATTGAACAGGTG | pEL78 |
| rEL-410 | TTGTTCATGGTATATCTCCTTTAAACAATGCGAAACGCATCGACTA | pEL78 |
| rEL-411 | TCTAAGCATGCAGGAGAAAGGTACCATGAATAAAGACACACTAATACCTACAACTAAAG | pEL79 |
| rEL-412 | TAAAGTTGTTCATGGTATATCTCCTTTAGCCGGCAAGTACACATCTTCTTTG | pEL79 |
| rEL-413 | GTATCTAAGCATGCAGGAGAAAGGTACCATGAATAATAATTTATTCGTGTCACCAGAAAC | pEL80 |
| rEL-414 | TAAAGTTGTTCATGGTATATCTCCTTTAGCCTACGAACACACACCTTCTTTGTC | pEL80 |
| rEL-455 | GCTGTGGTGATGATGGTGATGGCTGCTGCCCATGGTACCTTTCTCCTCTTTAATGAATTC | pEL90-96 |
| rEL-456 | CGCGTGCTAGAGGCATCAAATAAAAC | pEL90-96 |
| rEL-457 | ATCACCATCATCACCACAGCATGGCGGAAATCACCGGCGCGGGGA | pEL90 |
| rEL-458 | TTTGATGCCTCTAGCACGCGCTACCAGCGAATCAACGCCGCCCCCCA | pEL90 |
| rEL-459 | ATCACCATCATCACCACAGCATGTCCGATCCCATTCGTGTCCGCCT | pEL91 |
| rEL-460 | TTTGATGCCTCTAGCACGCGTTACATCCGGATAAGGGCGGATCCCCA | pEL91 |
| rEL-461 | ATCACCATCATCACCACAGCATGGAATTTTACGCCTCTCTTAAATCCATT | pEL92 |
| rEL-462 | TTTGATGCCTCTAGCACGCGCTAACTTCCTCCAAAATACACCAACGCT | pEL92 |
| rEL-463 | ATCACCATCATCACCACAGCATGAACGCAGGAATTTTAGGAGTAGGTAAA | pEL93 |
| rEL-464 | TTTGATGCCTCTAGCACGCGTTACTTACCCCAACGAATGATTAGGGC | pEL93 |
| rEL-467 | ATCACCATCATCACCACAGCATGCCGCGCGCCGCCGTGGTCT | pEL94 |
| rEL-468 | TTTGATGCCTCTAGCACGCGTCAGTCCATTGTCGGAACGATCTTC | pEL94 |
| rEL-469 | ATCACCATCATCACCACAGCATGTCCCCTACCGCCGCCGGTTCTT | pEL95 |
| rEL-470 | TTTGATGCCTCTAGCACGCGTCATGACGTCGTCCGTTCTCCTTGG | pEL95 |
| rEL-471 | ATCACCATCATCACCACAGCATGACCCGGGCGTCCGTGCTGACCG | pEL96 |
| rEL-472 | TTTGATGCCTCTAGCACGCGTCAGACCGGATCGACGGCGGGCCAG | pEL96 |
| rEL-148 | GGGAAAGGATCCATGAAAAATTGTGTCATCGTCAGTGCGG | N/A; atoB gene specific |
| rEL-149 | GGGAAAGCGGCCGCATTAATTCAACCGTTCAATCACCATCGC | N/A; atoB gene specific |
| rEL-157 | GGGAAAGCGGCCGCATTATTTTGAATAATCGTAGAAACCTTTTCCTG | N/A; crt.hbd fragment |
| | | specific |
| rEL-158 | GGGAAAGGATCCATGGAACTAAACAATGTCATCCTTGAAAAGGA | N/A; crt.hbd fragment |
| | | specific |
| rEL-160 | GGGAAAGGATCCATGATTGTAAAACCAATGGTTAGGAACAAT | N/A; T.d-ter gene specific |
| rEL-161 | GGGAAAGCGGCCGCATTAAATCCTGTCGAACCTTTCTACCTCG | N/A; T.d-ter gene specific |
| rEL-162 | GGGAAAGATCCATGAAAGTTACAAATCAAAAAGAACTAAAACAAAAGC | N/A; adhE2 gene specific |
| rEL-163 | GGGAAAGCGGCCGCATTAAAATGATTTTATATAGATATCCTTAAGTTCAC | N/A; adhE2 gene specific |
| rEL-323 | GGGAAAGGATCCGATGTCTGCGCAATCTCTCGAAGTTG | N/A; phaJ.phaB fragment |
| | | specific |
| rEL-326 | GGGAAAAAGCTTTTAACCCATGTGCAGACCACCGTTC | N/A; phaJ.phaB fragment |
| | | specific |
| rEL-349 | GGGAAAGAATTCGATGATTAAAGACACGCTAGTTTCTATAAC | N/A; bldh gene specific |
| rEL-350 | GGGAAAAAGCTTTTAACCGGCGAGTACACATCTTCTTTGTC | N/A; bldh gene specific |
| rEL-192 | AACAATTTCACACAGGAGATATACCATGGGCAGCAGCCATCACCATCATC | N/A; E.g.ter gene specific |
| rEL-203 | GTTTACAAGCATACTAGAGGATCGTTATTGTTGAGCGGCAGAAGGCAGATCC | N/A; E.g.ter gene specific |

Enzyme assays. Enzyme assays were conducted by using Bio-Tek PowerWave XS microplate spectrophotometer. Thiolase activity was measured via both condensation and thiolysis direction. The enzymatic reaction was monitored by the increase or decrease of absorbance at 303 nm which corresponded to the result of Mg²⁺ coordination with the diketo moiety of acetoacetyl-CoA. The enzymatic reaction was initiated by the addition of the enzyme. For purified enzyme reaction, the reaction mixture contained 100 mM Tris-HCl (pH 8.0), 20 mM MgCl₂, equimolar acetoacetyl-CoA and CoA. For the crude cyanobacteria extract assay, same buffer was used with 200 μM acetoacetyl-CoA and 300 μM CoA. Crude extract of strains EL22 (2.7 μg), EL14 (5.0 μg), and Wild-type (2.4 μg) were used for assay. Concentration of acetoacetyl-CoA was calculated based on a constructed standard curve.

Acetoacetyl-CoA synthase activity was measured by monitoring the increase of absorbance at 303 nm which corresponds to appearance of acetoacetyl-CoA. The reaction buffer is the same as that used for thiolase assay. Equimolar malonyl-CoA and acetyl-CoA were used for purified enzyme assay, while 400 pM of both malonyl-CoA and acetyl-CoA were used for crude extract assay. Crude extract of strains EL22 (27 μg), EL14 (50 μg), and Wild-type (24 μg) were used for assay.

Production of 1-butanol. A loopful of S. elongates 7942 was used to inoculate fresh 50 mL BG-11. 500 mM IPTG was used to induce the growing culture at cell density OD730 of 0.4 to 0.6 with 1 mM IPTG as final concentration. 5 mL of growing culture was sampled for cell density and 1-butanol production measurements every two days for up to day 8 after which sampling time was switched to every three days. After sampling, 5 mL of fresh BG-11 with 50 mM NaHCO₃, appropriate antibiotics, and IPTG were added back to the culture.

1-Butanol quantification. Culture samples (5 mL) were centrifuged for 20 minutes at 5,250×g. The supernatant (900 μL) was then mixed with 0.1% v/v 2-methyl-pentanol (100 μL) as internal standard. The mixture was then vortexed and directly analyzed on Agilent GC 6850 system with flame ionization detector and DB-'FAP capillary column (30 m, 0.32 mm i.d., 0.25 film thickness) from Agilent Technologies (Santa Clara, Calif.). 1-Butanol in the sample was identified and quantified by comparing to 0.001% v/v 1-butanol standard. 1-Butanol standard of 0.001% v/v was prepared by 100-fold dilution of a 0.1% v/v solution. The GC result was analyzed by Agilent software Chem Station (Rev.B.04.01 SP1). Amount of 1-butanol in the sample was then calculated based on the ratio of its integrated area and that of the 0.001% 1-butanol standard.

Helium gas was used as the carrier gas with 9.52 psi inlet pressure. The injector and detector temperatures were maintained at 225° C. Injection volume was 1 μL. The GC oven temperature was initially held at 85° C. for 3 minutes and then raised to 235° C. with a temperature ramp of 45° C./min. The GC oven was then maintained at 235° C. for 1 minute before completion of analysis. Column flow rate was 1.7 ml/min.

Alcohol production by E. coli expressing butyraldehyde dehydrogenase. E. coli wild type is based on strain BW25113Transformed E. coli strain JCL299 (ΔadhE, ΔldhA, Δfrd, Δpta) was selected on LB plate supplemented with ampicillin (100 μg/mL) and kanamycin (50 μg/mL). Three colonies were picked from the plate to make overnight pre-culture. The pre-cultures were then used to inoculate Terrific broth (TB; 12 g tryptone, 24 g yeast extract, 2.31 g KH₂PO₄, 12.54 g K₂HPO₄, 4 mL glycerol per liter of water) supplemented with 20 g/L glucose. Culture sample (2 mL) was centrifuged for 5 minutes at 21,130×g. The supernatant was analyzed by GC following the same method as that described in section 2.8.

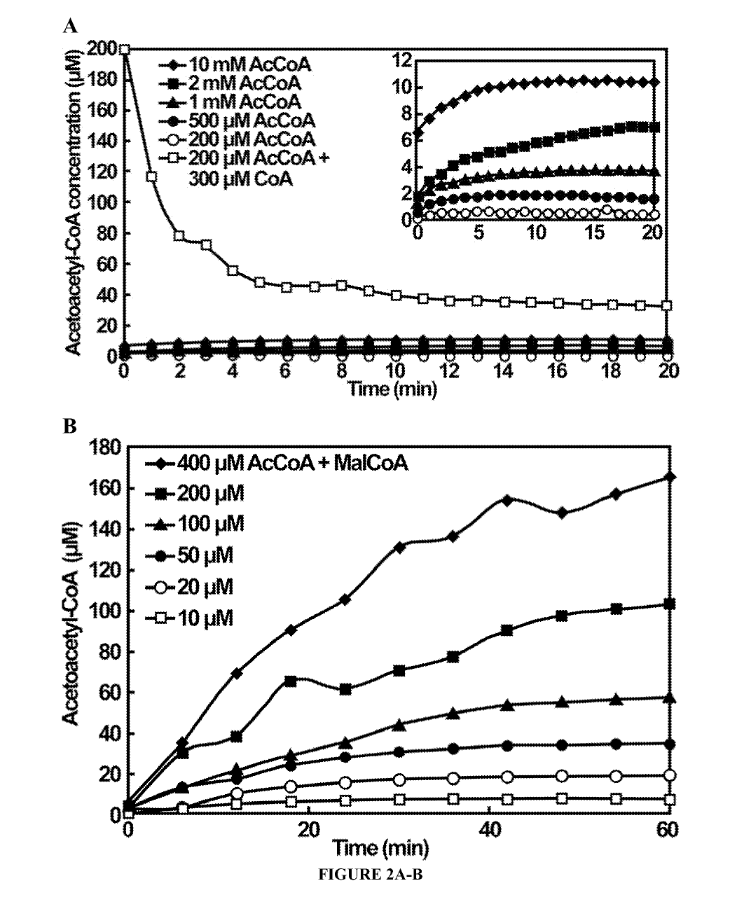

Incorporating synthetic driving force for 1-butanol biosynthetic pathway design. We hypothesized that insufficient carbon flux into the pathway led to the difficulty to synthesize 1-butanol under aerobic photosynthetic condition. The first step of the pathway, catalyzed by thiolase, is readily reversible and strongly favors the formation of reactants. Using purified AtoB in spectrophotometric assay, we demonstrated that condensation reaction is unfavorable (FIG. 2A) with an equilibrium constant at pH 8.0 of (1.1±0.2)×10⁻⁵, corresponding to ΔG° of 6.8 kcal/mol and consistent with previous literature value (19). Therefore, without sufficient carbon flux to acetyl-CoA or an efficient product trap, there is no driving force for the formation of acetoacetyl-CoA.

We searched for alternative pathways that drive the formation of acetoacetyl-CoA. We investigated into metabolic pathways that share similarities with the CoA 1-butanol pathway, including fatty acid synthesis, polyketide synthesis, and β-oxidation. In particular, fatty acid synthetic pathway is almost identical to CoA 1-butanol pathway with two exceptions. First exception is that fatty acid synthesis utilizes acyl-carrier protein (ACP) instead of CoA as the thioester recognition moiety. Second is that fatty acid biosynthesis also requires the activation of acetyl-CoA into malonyl-CoA. Malonyl-CoA is synthesized from acetyl-CoA, HCO₃⁻, and ATP by acetyl-CoA carboxylase (Acc). The formation of malonyl-CoA is effectively irreversible due to ATP hydrolysis. Malonyl-CoA is then converted into malonyl-ACP and acts as the carbon addition unit for fatty acid synthesis. Ketoacyl-ACP synthase III (KAS III) catalyzes the irreversible condensation of malonyl-ACP and acetyl-CoA to synthesize the four carbon intermediate 3-ketobutyryl-ACP, equivalent in structure to acetoacetyl-CoA with different thioester recognition marker.

We therefore hypothesized that utilizing the energy release from ATP hydrolysis (ΔG°′ of −7.3 kcal/mol) (20) would compensate for the energy require for condensation of acetyl-CoA into acetoacetyl-CoA. By combining the reaction catalyzed by thiolase with ATP hydrolysis, we expected a net reaction that is thermodynamically favored (ΔG°′<0). More importantly, CO₂ release from the decarboxylative condensation drives the formation of acetoacetyl-CoA as gaseous CO₂ leaves the system, shifting the reaction towards the product. Fatty acid and polyketide chain elongation have naturally evolved this mechanism to enable this thermodynamically unfavorable reaction and elongate carbon chain length. We therefore reasoned that by taking advantage of this evolved mechanism, we can push the carbon flux into our desired CoA 1-butanol pathway. Furthermore, this mechanism may be especially useful for photoautotrophs that readily produce ATP from light energy.

We bioprospected for KASIII that utilize malonyl-CoA rather than malonyl-ACP for condensation with acetyl-CoA. Since both ACP and CoA carry the phosphopantetheine moiety which forms thioester bond with the malonyl-moiety of malonyl-CoA, KASIII and KASIII-like enzymes may be able to react with malonyl-CoA. We started by cloning a variety of KASIII and KASIII-like enzymes from different organisms. We then tested their expression in E. coli (SI FIG. 1) and assayed their activity towards condensing malonyl-CoA with acetyl-CoA after His-tag purification (Table 1). Of the enzymes tested, NphT7 (21) was the most active (specific activity of 6.02 umol/min/mg). Other enzymes such as Bamb6244, GOX0115, and PAE-FabH2 were also active while the rest showed no detectable activity. As Shown in FIG. 2B, condensation reaction catalyzed by NphT7 using malonyl-CoA and acetyl-CoA is irreversible and accumulates acetoacetyl-CoA as the product. At low starting concentration of malonyl-CoA and acetyl-CoA, conversion to acetoacetyl-CoA is higher than high starting substrate concentration. This result is most likely due to depletion of malonyl-CoA as NphT7 also catalyzes malonyl-CoA self reaction.

TABLE 1

| Enzyme | Specific activity (umol/min/mg) | |
|---|---|---|
| Bamb6244 | 0.0116 ± 0.0002 | |
| GOX0115 | 0.0099 ± 0.0011 | |
| HP0202 | n.d | |
| Imo2202 | n.d | |
| PAE-FabH2 | 0.0140 ± 0.0010 | |
| SAV-FabH4 | n.d | |
| SCO5858 | n.d | |
| NphT7 | 6.02 | 0.25 |

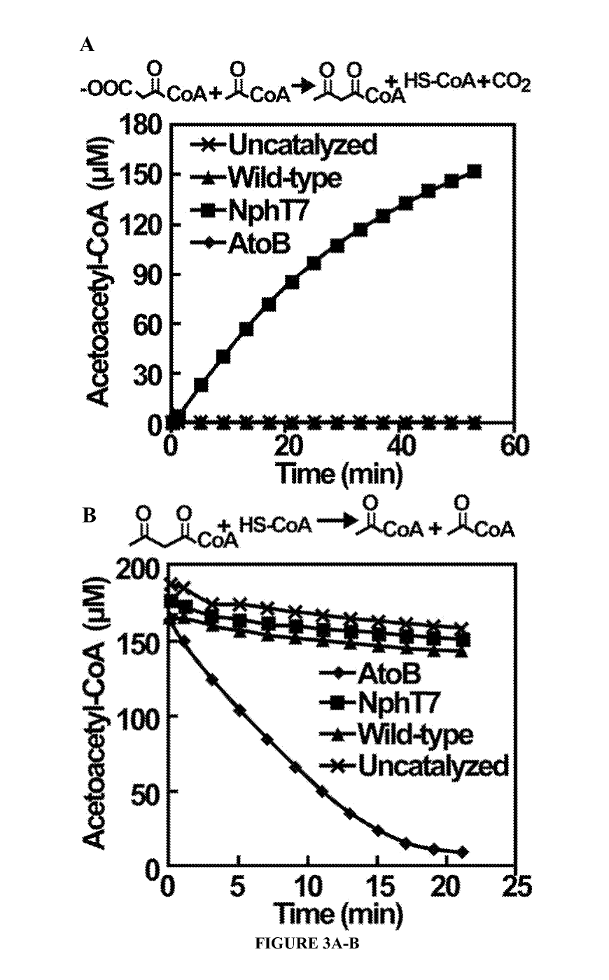

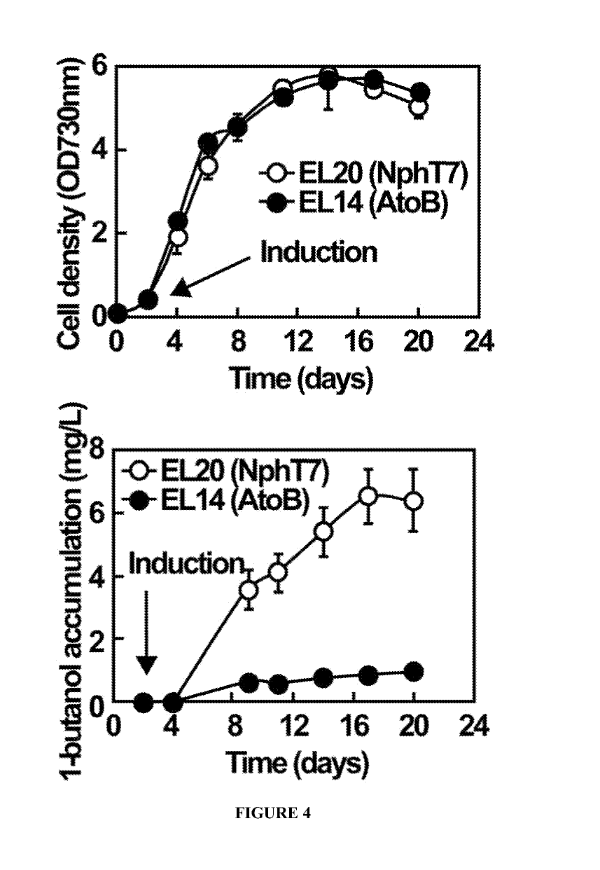

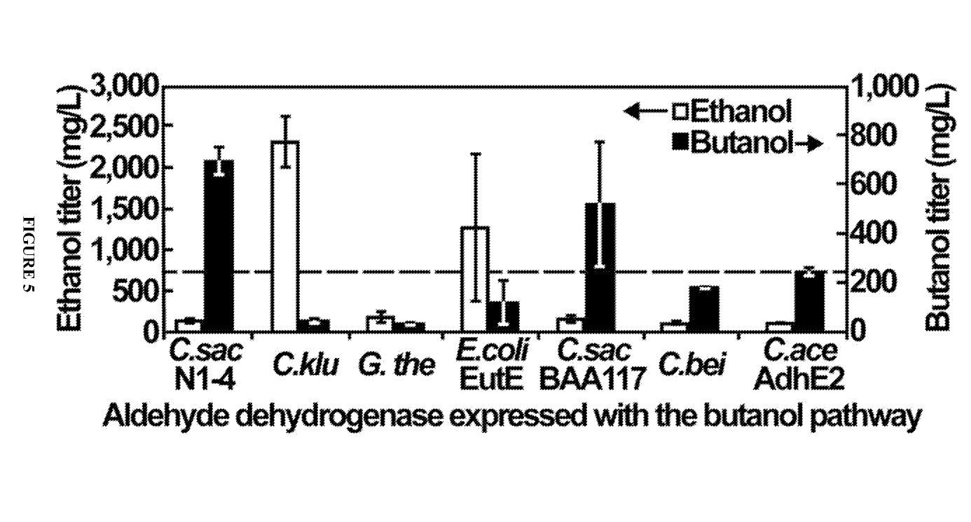

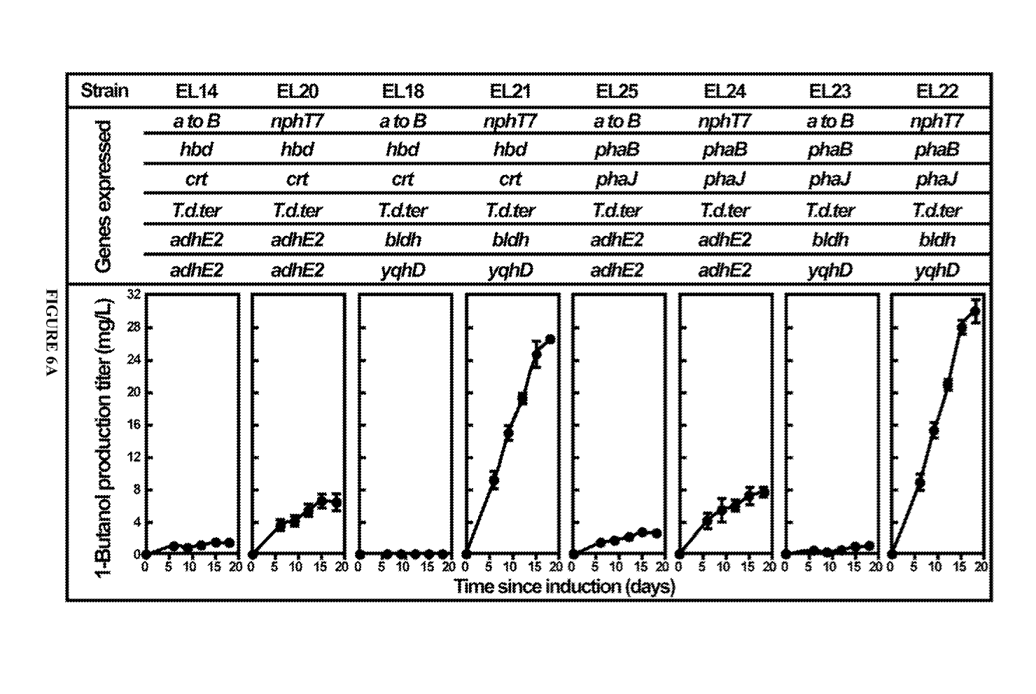

Expression of Acetoacetyl-CoA synthase enables photosynthetic production of 1-butanol. To test our hypothesis that increasing driving force will push carbon flux into the CoA pathway, we integrated this synthetic driving force into S. elongatus PCC 7942. The gene nphT7 was synthesized and recombined into the genome of S. elongatus PCC 7942 along with the other genes of the CoA 1-butanol pathway (hbd, crt, Td.ter, and adhE2), resulting in strain EL20. As shown in (FIG. 3A), crude extract from strain EL20 expressing NphT7 was able to catalyze formation of acetoacetyl-CoA by condensation of malonyl-CoA and acetyl-CoA and was not capable of catalyzing the thiolysis of acetoacetyl-CoA. On the other hand, crude extract from strain EL14 expressing AtoB catalyzed thiolysis much more efficient than the condensation reaction (FIG. 3B). The two strains EL20 and EL14 share nearly identical growth rate (FIG. 4A). However, Strain EL20 produced 6.5 mg/L (FIG. 4B) of 1-butanol while Strain EL14 produced only trace amounts of 1-butanol (FIG. 4C). This result indicated that ATP driven acetoacetyl-CoA formation is more efficient at capturing carbon flux into the CoA 1-butanol pathway.

Substitution of NADPH utilizing enzymes aids 1-butanol production. Cyanobacteria produce NADPH as the direct result of photosynthesis. Intracellular NAD⁺ and NADP⁺ level differ by ratio of about 1:10 (22) in S. elongatus 7942. Thus NADH utilizing pathway may be unfavorable in cyanobacteria. The CoA 1-butanol pathway requires four NADH per 1-butanol produced. Changing the cofactor preference of this pathway may aid the production of 1-butanol.

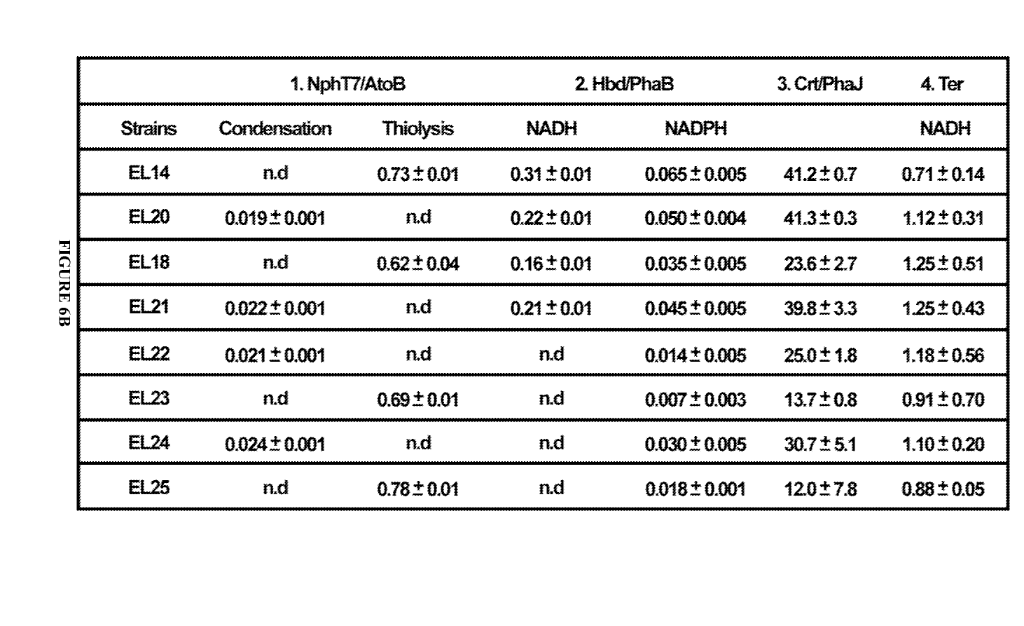

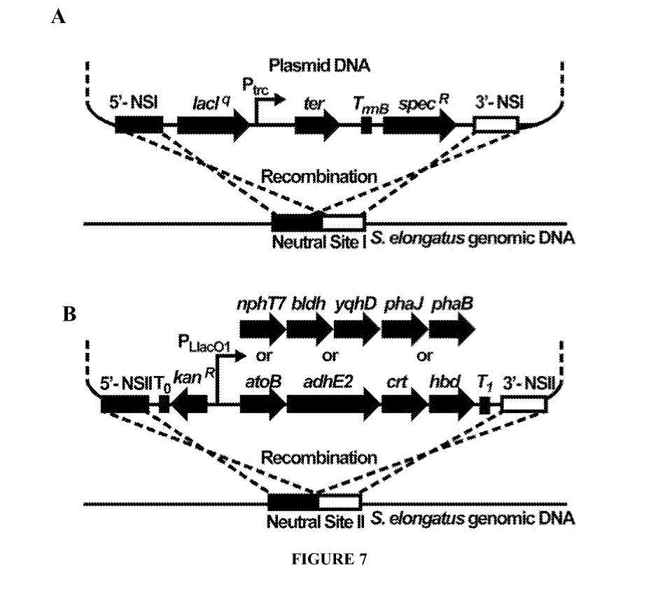

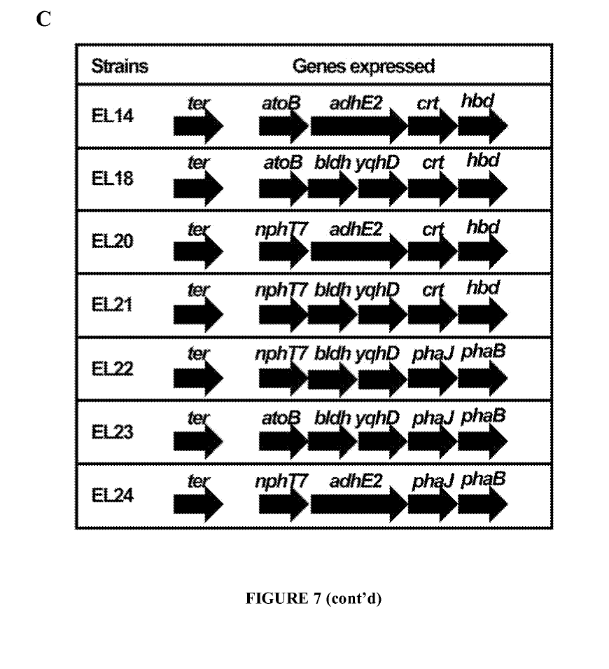

As depicted in FIG. 1 (outlined in red), we identified enzymes that utilize NADPH or both NADPH and NADH by bioprospecting. NADP-dependent alcohol dehydrogenase (YqhD) (23) from E. coli has been demonstrated to aid production of higher chain alcohols (18, 24). YqhD is a good replacement candidate for the alcohol dehydrogenase domain of AdhE2. To couple to YqhD, we needed a CoA-acylating butyraldehyde dehydrogenase to replace the aldehyde dehydrogenase domain of AdhE2. We bioprospected for enzymes catalyzing reduction of butyryl-CoA to butyraldehyde. CoA-acylating butyraldehyde dehydrogenase (Bldh) is found in high butanol producing Clostridium species including C. beijerinckii NCIMB 8052 (25), C. saccharobutylicum ATCC BAA-117, and C. saccharooerbutylacetonicum NI-4 (26). In particular, Bldh from C. beijerinckii (27) has been purified and demonstrated activity in vitro with both NADH and NADPH as reducing cofactor. Based on sequence homology of Bldh from C. beijerinckii, we cloned additional Bldh-like enzymes from various organisms including C. saccharoperbutylacetbnicum NI-4, C. saccharobutylicum ATCC BAA-117, Geobacillus thermoglucosidasius, Clostridium Kluyveri, and E. coli. We assessed the performance of these Bldh by 1-butanol production in recombinant E. coli. As shown in FIG. 5, E. coli strain expressing C. saccharoperbutylacetonicum NI-4 Bldh along with rest of the CoA 1-butanol pathway produced the highest titer of 1-butanol, exceeding the 1-butanol produced by E. coli strain expressing AdhE2 by nearly 3-fold. Therefore C. saccharoperbutylacetonicum NI-4 bldh and E. coli yqhD were cloned and expressed in S. elongatus PCC 7942 to replace adhE2. As results shown in FIG. 6, the production of 1-butanol from strain EL21 expressing bldh and yqhD (26.5 mg/L) exceeded that from strain EL20 expressing adhE2 by around 400%. This result corresponded to the same observation seen in recombinant E. coli. The increase in 1-butanol production by expression of bldh and adhE2 may be attributed to higher activity or expression of Bldh and YqhD in comparison to AdhE2 as well as the.ability to utilize NADPH.

To further investigate the effect of changing cofactor dependence from NADH to NADPH, Acetoacetyl-CoA reductase (PhaB) (28, 29) was used to replace Hbd. PhaB from Ralstonia eutropha is an enzyme found in the poly-hydroxyalkanoate biosynthetic pathway for reducing 3-ketobutyryl-CoA to 3-hydroxybutyryl-CoA using NADPH. However, PhaB produces the (R)-stereoisomer of 3-hydroxybutyryl-CoA instead of the (S)-stereoisomer produced by Hbd. As a result, Crt cannot be used for the subsequent dehydration to produce crotonyl-CoA. Upon reaction of (R)-3-hydroxybutyryl-CoA with Crt, isocrotonyl-CoA is produced (30) and cannot be further reduced by Ter. Therefore, a different crotonase capable of reacting with (R)-3-hydroxybutyryl-CoA is necessary in order to utilize PhaB for the reduction of acetoacetyl-CoA. (R)-specific enoyl-CoA hydratase (PhaJ) (31) is found in Aeromonas caviae and is responsible for diverging p-oxidation intermediates into production of poly-hydroxyalkanoates. PhaJ dehydrates (R)-3-hydroxybutyryl-CoA into crotonyl-CoA, and therefore it couples to PhaB for the reduction of 3-ketobutyryl-CoA. Genes phaB and phaJ were codon optimized for expression in S. elongatus 7942. We integrated the genes phaB and phaJ into S. elongatus 7942 to replace hbd and crt. As shown in FIG. 6, the effect of this replacement is minimal. 1-Butanol production from strains EL22 (29.9 mg/L) and EL24 (7.7 mg/L) expressing PhaB and PhaJ only slightly outperformed strains EL21 (26.5 mg/L) and EL20 (6.4 mg/L) expressing Hbd and Crt.

Direct 1-butanol production from cyanobacteria under oxygenic condition is desirable because it may be developed into a continuous process and reduces the number of processing steps. Metabolic engineering of cyanobacteria has enabled the production of Isobutyraldehyde, isobutanol (18), 1-butanol (17), ethanol (32, 33), ethylene (34), isoprene (35), sugars, lactic acid (36), fatty alcohols (37), and fatty acids (38) from CO₂. The pathways for the high flux production of isobutanol and ethanol naturally have decarboxylation as driving force. The loss of CO₂ is often considered as irreversible. In contrast, the CoA pathway utilizing thiolase does not have such significant driving force. Although this pathway enables production in E. coli under fermentative conditions, cyanobacteria are different in their metabolism. The same pathway would require additional engineering to function according to host. Under light condition, cyanobacteria readily generate ATP from photosynthesis. Therefore, consumption of ATP to enhance thermodynamic favorability of the CoA pathway is an effective approach. We changed the nature of the CoA pathway from a fermentative pathway into a biosynthetic pathway. Our strategy models fatty acid and polyketide synthesis where decarboxylative condensation of malonyl-CoA with acetyl-CoA serves as an irreversible trap for elongation of carbon chain.

Reducing cofactor preference is an important aspect of pathway design. Depending on the production condition and organisms' natural metabolism, changing cofactor preference is necessary to achieve high flux production. For example, changing isobutanol production pathway into utilizing NADH increases the productivity and yield under anaerobic condition in recombinant E. coli (39). In contrast, pathways utilizing NADPH is preferred in cyanobacteria because NADPH is more abundant. By utilizing NADPH dependent enzymes, our 1-butanol production enhanced from 6.5 mg/L to 29.9 mg/L (FIG. 6A). Current limitation may be the synthesis of malonyl-CoA. Compared to the high flux production of isobutanol and isobutyrldehyde in cyanobacteria, the carbon flux through our 1-butanol pathway is suboptimal. Malonyl-CoA biosynthesis is considered as the limiting step in fatty acid biosynthesis (40). Therefore, increasing carbon flux towards the synthesis of acetyl-CoA and malonyl-CoA may be necessary to increase 1-butanol production. Intracellular acetyl-CoA and malonyl-CoA supply may be increased by increasing CoA biosynthesis (41), overexpression of Acc (42-45), phosphoglycerate kinase (Pgk), glyceraldehyde-3-phosphate dehydrogenase (Gapd) (46), and inhibition of fatty acid biosynthesis (47). Combining these approaches, our malonyl-CoA dependent 1-butanol pathway is expected to achieve higher production.

To our knowledge, this is the first example of recombinant 1-butanol production utilizing Bldh. Expression of Bldh alone would enable the production of butyraldehyde. Similar to the production of isobutyraldehyde, butyraldehyde has a lower vapor pressure and solubility compared to 1-butanol. Therefore product removal by gas stripping is faster and thereby lowering product toxicity. Butyraldehyde is also a useful chemical with annual Consumption of around 1,200,000 tons in the U.S. (48). Furthermore, butyraldehyde is an important intermediate in the chemical production of 2-ethylhexanol, a widely used chemical for producing plasticizer with world-wide annual production of 2,600,000 tons (48).

A number of embodiments of the invention have been described. Nevertheless, it will be understood that various modifications may be made without departing from the spirit and scope of the invention. Accordingly, other embodiments are within the scope of the following claims.

### REFERENCES (INCORPORATED HEREIN BY REFERENCE)

1. Shen CR & Liao J C (2008) Metabolic engineering of Escherichia coli for 1-butanol and 1-propanol production via the keto-acid pathways. Metab. Eng. 10(6):312-320.

2. Atsumi S & Liao J C (2008) Directed Evolution of Methanococcus jannaschii Citramalate Synthase for Biosynthesis of 1-Propanol and 1-Butanol by Escherichia coli. Appl. Environ. Microbiol. 74(24):7802-7808.

3. Dekishima Y, Lan E I, Shen C R, Cho K M, & Liao J C (2011) Extending Carbon Chain Length of 1-Butanol Pathway for 1-Hexanol Synthesis from Glucose by Engineered Escherichia coli. J. Am. Chem. Soc. 133(30):11399-11401.

4. Dellomonaco C, Clomburg J M, Miller E N, & Gonzalez R (2011) Engineered reversal of the beta-oxidation cycle for the synthesis of fuels and chemicals. Nature 476(7360):355-359.

5. Nicolaou S A, Gaida S M, & Papoutsakis E T (2010) A comparative view of metabolite and substrate stress and tolerance in microbial bioprocessing: From biofuels and chemicals, to biocatalysis and bioremediation. Metab. Eng. 12(4):307-331.

6. Jiang Y, et al. (2009) Disruption of the acetoacetate decarboxylase gene in solvent-producing Clostridium acetobutylicum increases the butanol ratio. Metab. Eng. 11(4-5):284-291.

7. Sillers R, Chow A, Tracy B, & Papoutsakis E T (2008) Metabolic engineering of the non-sporulating, non-solventogenic Clostridium acetobutylicum strain M5 to produce butanol without acetone demonstrate the robustness of the acid-formation pathways and the importance of the electron balance. Metab. Eng. 10(6):321-332.

8. Yu M R, Zhang Y L, Tang I C, & Yang S T (2011) Metabolic engineering of Clostridium tyrobutyricum for n-butanol production. Metab. Eng. 13(4):373-382.

9. Atsumi S, et al. (2008) Metabolic engineering of Escherichia coli for 1-butanol production. Metab. Eng. 10(6):305-311.

10. Inui M, et al. (2008) Expression of Clostridium acetobutylicum butanol synthetic genes in Escherichia coli. Appl. Microbiol. Biotechnol. 77(6):1305-1316.

11. Nielsen D R, et al. (2009) Engineering alternative butanol production platforms in heterologous bacteria. Metab. Eng. 11(4-5):262-273.

12. Berezina O V, et al. (2010) Reconstructing the clostridial n-butanol metabolic pathway in Lactobacillus brevis. Appl. Microbiol. Biotechnol. 87(2):635-646.

13. Steen E J, et al. (2008) Metabolic engineering of Saccharomyces cerevisiae for the production of n-butanol. Microbial Cell Factories 7(36).

14. Boynton Z L, Bennett G N, & Rudolph F B (1996) Cloning, sequencing, and expression of clustered genes encoding beta-hydroxybutyryl-coenzyme A (CoA) dehydrogenase, crotonase, and butyryl-CoA dehydrogenase from Clostridium acetobutylicum ATCC 824. J. Bacteriol. 178(11):3015-3024.

15. Li F, et al. (2008) Coupled ferredoxin and crotonyl coenzyme a (CoA) reduction with NADH catalyzed by the butyryl-CoA dehydrogenase/Etf complex from Clostridium kluyveri. J. Bacteriol. 190(3):843-850.

16. Shen C R, et al. (2011) Driving forces enable high-titer anaerobic 1-butanol synthesis in Escherichia coli. Appl. Environ. Microbiol. 77(9):2905-2915.

17. Lan E I & Liao J C (2011) Metabolic engineering of cyanobacteria for 1-butanol production from carbon dioxide. Metab. Eng. 13(4):353-363.

18. Atsumi S, Higashide W, & Liao J C (2009) Direct photosynthetic recycling of carbon dioxide to isobutyraldehyde. Nat. Biotechnol. 27(12):1177-1180.

19. Stern J R, Coon M J, & Delcampillo A (1953) Acetoacetyl Coenzyme-a as Intermediate in the Enzymatic Breakdown and Synthesis of Acetoacetate. J. Am. Chem. Soc. 75(6):1517-1518.

20. Fuchs G (2011) Alternative pathways of carbon dioxide fixation: insights into the early evolution of life? Annu. Rev. Microbiol. 65:631-658.

21. Kuzuyama T, Okamura E, Tomita T, Sawa R, & Nishiyama M (2010) Unprecedented acetoacetyl-coenzyme A synthesizing enzyme of the thiolase superfamily involved in the mevalonate pathway. Proc. Natl. Acad. Sci. U.S.A. 107(25):11265-11270.

22. Tamoi M, Miyazaki T, Fukamizo T, & Shigeoka S (2005) The Calvin cycle in cyanobacteria is regulated by CP12 via the NAD(H)/NADP(H) ratio under light/dark conditions. Plant J. 42(4):504-513.

23. Perez J M, Arenas F A, Pradenas G A, Sandoval J M, & Vasquez C C (2008) Escherichia coli YqhD exhibits aldehyde reductase activity and protects from the harmful effect of lipid peroxidation-derived aldehydes. J. Biol. Chem. 283(12):7346-7353.

24. Atsumi S, et al. (2010) Engineering the isobutanol biosynthetic pathway in Escherichia coli by comparison of three aldehyde reductase/alcohol dehydrogenase genes. Appl. Microbiol. Biotechnol. 85(3):651-657.

25. Toth J, Ismaiel A A, & Chen J S (1999) The ald gene, encoding a coenzyme A-acylating aldehyde dehydrogenase, distinguishes Clostridium beijerinckii and two other solvent-producing clostridia from Clostridium acetobutylicum. Appl. Environ. Microbiol. 65(11):4973-4980.

26. Kosaka T, Nakayama S, Nakaya K, Yoshino S, & Furukawa K (2007) Characterization of the sol operon in butanol-hyperproducing Clostridium saccharoperbutylacetonicum strain N1-4 and its degeneration mechanism. Biosci Biotech Bioch 71(1):58-68.

27. Yan R T & Chen J S (1990) Coenzyme a-Acylating Aldehyde Dehydrogenase from Clostridium-Beijerinckii Nrrl-B592. Appl. Environ. Microbiol. 56(9):2591-2599.

28. Slater S C, Voige W H, & Dennis D E (1988) Cloning and Expression in Escherichia-Coli of the Alcaligenes-Eutrophus H16 Poly-Beta-Hydroxybutyrate Biosynthetic-Pathway. J. Bacteriol. 170(10):4431-4436.

29. Peoples O P & Sinskey A J (1989) Poly-Beta-Hydroxybutyrate (Phb) Biosynthesis in Alcaligenes-Eutrophus H16 -Identification and Characterization of the Phb Polymerase Gene (Phbc). J. Biol. Chem. 264(26):15298-15303.

30. Bond-Watts B B, Bellerose R J, & Chang M C Y (2011) Enzyme mechanism as a kinetic control element for designing synthetic biofuel pathways. Nat. Chem. Biol. 7(4):222-227.

31. Fukui T, Shiomi N, & Doi Y (1998) Expression and characterization of (R)-specific enoyl coenzyme A hydratase involved in polyhydroxyalkanoate biosynthesis by Aeromonas caviae. J. Bacteriol. 180(3):667-673.

32. Deng M D & Coleman J R (1999) Ethanol synthesis by genetic engineering in cyanobacteria. Appl. Environ. Microbiol. 65(2):523-528.

33. Dexter J & Fu P C (2009) Metabolic engineering of cyanobacteria for ethanol production. Energy & Environmental Science 2(8):857-864.

34. Takahama K, Matsuoka M, Nagahama K, & Ogawa T (2003) Construction and analysis of a recombinant cyanobacterium expressing a chromosomally inserted gene for an ethylene-forming enzyme at the psbAl locus. J Biosci Bioeng 95(3):302-305.

35. Lindberg P, Park S, & Hells A (2010) Engineering a platform for photosynthetic isoprene production in cyanobacteria, using Synechocystis as the model organism. Metab. Eng. 12(1):70-79.

36. Niederholtmeyer H, Wolfstadter B T, Savage D F, Silver P A, & Way J C (2010) Engineering Cyanobacteria To Synthesize and Export Hydrophilic Products. Appl. Environ. Microbiol. 76(11):3462-3466.

37. Tan X M, et al. (2011) Photosynthesis driven conversion of carbon dioxide to fatty alcohols and hydrocarbons in cyanobacteria. Metab. Eng. 13(2):169-176.

38. Liu X Y, Sheng J, & Curtiss R (2011) Fatty acid production in genetically modified cyanobacteria. Proc. Natl. Acad. Sci. U.S.A. 108(17):6899-6904.

39. Bastian S, et al. (2011) Engineered ketol-acid reductoisomerase and alcohol dehydrogenase enable anaerobic 2-methylpropan-1-ol production at theoretical yield in Escherichia coli. Metab. Eng. 13(3):345-352.

40. Davis M S, Solbiati J, & Cronan J E (2000) Overproduction of acetyl-CoA carboxylase activity increases the rate of fatty acid biosynthesis in Escherichia coli. J. Biol. Chem. 275(37):28593-28598.

41. Vadali R V, Bennett G N, & San K Y (2004) Cofactor engineering of intracellular CoA/acetyl-CoA and its effect on metabolic flux redistribution in Escherichia coli. Metab. Eng. 6(2):133-139.

42. Leonard E, Lim K H, Saw P N, & Koffas M A G (2007) Engineering central metabolic pathways for high-level flavonoid production in Escherichia coli. Appl. Environ. Microbiol. 73(12):3877-3886.

43. Zha W J, Rubin-Pitel S B, Shao Z Y, & Zhao H M (2009) Improving cellular malonyl-CoA level in Escherichia coli via metabolic engineering. Metab. Eng. 11(3):192-198.

44. Miyahisa I, et al. (2005) Efficient production of (2S)-flavanones by Escherichia coli containing an artificial biosynthetic gene cluster. Appl. Microbiol. Biotechnol. 68(4):498-504.

45. Lu X F, Vora H, & Khosla C (2008) Overproduction of free fatty acids in E. coli: Implications for biodiesel production. Metab. Eng. 10(6):333⁻339.

46. Xu P, Ranganathan S, Fowler Z L, Maranas C D, & Koffas M A G (2011) Genome-scale metabolic network modeling results in minimal interventions that cooperatively force carbon flux towards malonyl-CoA. Metab. Eng. 13(5):578-587.

47. Santos C N S, Koffas M, & Stephanopoulos G (2011) Optimization of a heterologous pathway for the production of flavonoids from glucose. Metab. Eng. 13(4):392-400.

48. Kohlpaintner C, Schulte M, Falbe J, Lappe P, & Weber J (2000) Aldehydes, Aliphatic. Ullmann's Encyclopedia of Industrial Chemistry, (Wiley-VCH Verlag GmbH & Co. KGaA).

49. Bustos S A & Golden S S (1991) Expression of the Psbdii Gene in Synechococcus Sp Strain-Pcc-7942 Requires Sequences Downstream of the Transcription Start Site. J. Bacteriol. 173(23):7525-7533.

50. Bustos S A & Golden S S (1992) Light-Regulated Expression of the Psbd Gene Family in Synechococcus-Sp Strain Pcc-7942—Evidence for the Role of Duplicated Psbd Genes in Cyanobacteria. Mol. Gen. Genet. 232(2):221-230.

51. Andersson C R, et al. (2000) Application of bioluminescence to the study of circadian rhythms in cyanobacteria. Bioluminescence and Chemiluminescence, Pt C 305:527-542.

52. Gibson D G, et al. (2009) Enzymatic assembly of DNA molecules up to several hundred kilobases. Nat. Methods 6(5):343-345.

53. Nishimura T, Saito T, & Tomita K (1978) Purification and properties of beta-ketothiolase from Zoogloea ramigera. Arch. Microbiol. 116(1):21-27.

54. Datsenko K A & Wanner B L (2000) One-step inactivation of chromosomal genes in Escherichia coli K-12 using PCR products. Proc. Natl. Acad. Sci. U.S.A. 97(12):6640-6645.

1. A recombinant photoautotroph or photoheterotroph microorganism that produces 1-butanol wherein the alcohol is produced through a malonyl-CoA dependent pathway.

2. The recombinant photoautotroph or photoheterotroph microorganism of claim 1, wherein the microorganism comprises expression or elevated expression of an enzyme that converts acetyl-CoA to malonyl-CoA, malonyl-CoA to Acetoacetyl-CoA, and at least one enzyme that converts (a) acetoacetyl-CoA to (R)- or (S)-3-hydroxybutyryl-CoA and (R)- or (S)-3-hydroxybutyryl-CoA to crotonyl-CoA, crotonyl-CoA to butyryl-CoA, butyryl-CoA to butyraldehyde and butyraldehyde to 1-butanol.

3. The recombinant microorganism of claim 1, wherein the microorganism comprises a metabolic pathway for the production of 1-butanol that is an NADPH dependent pathway.

4. The recombinant microorganism of claim 1, wherein the photoautotrophic or photoheterotrophic microorganism is engineered to express or overexpress one or more polypeptides that convert acetyl-CoA to Malonyl-CoA and malonyl-CoA to Acetoacetyl-CoA.

5. The recombinant microorganism of claim 4, wherein the one or more polypeptides comprises a nphT7 polypeptide comprising at least 90% identity to SEQ ID NO:18 and having acetoacetyl-CoA synthase activity.

6. The recombinant microorganism of claim 1, wherein the recombinant microorganism is engineered to express an acetyl-CoA carboxylase.

7. The recombinant microorganism of claim 6, wherein the acetyl-CoA carboxylase comprises a sequence that is at least 90% identical to SEQ ID NO:2.

8. The recombinant microorganism of claim 4, wherein the microorganism further expresses or overexpresses one or more enzymes that carries out a metabolic function selected from the group consisting of (a) converting acetoacetyl-CoA to (R)-3-hydroxybutyryl-CoA, (b) converting acetoacetyl-CoA to (S)-3-hydroxybutyryl-CoA, (c) converting (R)-3-hydroxybutyryl-CoA to crotonyl-CoA, (d) converting (S)-3-hydroxybutyryl-CoA to crotonyl-CoA, (e) converting crotonyl-CoA to butyryl-CoA, (f₁) converting butyryl-CoA to butyraldehyde and butyraldehyde to 1-butanol, or (f₂) butyrl-CoA to 1-butanol.

9. The recombinant microorganism of claim 8, wherein the recombinant microorganism comprises an NADPH dependent metabolic pathway that converts (i) acetyl-CoA to malonyl-CoA, (ii) malonyl-CoA to acetoacetyl-CoA, (iii) acetoacetyl-CoA to (R)-3-hydroxybutyryl-CoA, (iv) (R)-3-hydroxybutyryl-CoA to crotonyl-CoA, (v) crotonyl-CoA to butyryl-CoA, (vi) butyryl-CoA to butyraldehyde, and (vii) butyraldehyde to 1-butanol.

10. The recombinant microorganism of claim 8, wherein the recombinant microorganism comprises a NADH dependent metabolic pathway that converts (i) acetyl-CoA to malonyl-CoA, (ii) malonyl-CoA to acetoacetyl-CoA, (iii) acetoacetyl-CoA to (S)-3-hydroxybutyryl-CoA, (iv) (S)-3-hydroxybutyryl-CoA to crotonyl-CoA, (v) crotonyl-CoA to butyryl-CoA, and (vi) butyryl-CoA to 1-butanol.

11. The recombinant microorganism of claim 8, wherein the recombinant microorganism comprises an NADPH dependent metabolic pathway that converts (i) acetyl-CoA to acetoacetyl-CoA, (ii) acetoacetyl-CoA to (R)-3-hydroxybutyryl-CoA, (iii) (R)-3-hydroxybutyryl-CoA to crotonyl-CoA, (iv) crotonyl-CoA to butyryl-CoA, (v) butyryl-CoA to butyraldehyde, and (vi) butyraldehyde to 1-butanol.

12. The recombinant microorganism of 8, wherein the microorganism is a photoautotrophic or photoheterotrophic microorganism that is engineered to express or overexpress an acetyl-CoA carboxylase and an acetoacetyl-CoA synthase.

13. The recombinant microorganism of claim 12, wherein the microorganism further expresses or overexpresses one or more enzymes selected from the group consisting of (a) acetoacetyl-CoA reductase, (b) enoyl-CoA hydratase, (c) crotonyl-CoA reductase, and (d) an alcohol/aldehyde dehydrogenase.

14. The recombinant microorganism of claim 8, wherein the microorganism comprises a photoautotrophic or photoheterotrophic organism that is engineered to express or overexpress an acetyl-CoA carboxylase and an acetoacetyl-CoA synthase and further expresses or overexpresses one or more enzymes selected from the group consisting of (a) acetoacetyl-CoA reductase, (b) enoyl-CoA hydratase, (c) trans-2-enoyl-CoA reductase, and (d) an alcohol/aldehyde dehydrogenase.

15. The recombinant microorganism of claim 8, wherein the microorganism comprises a photoautotrophic or photoheterotrophic organism that is engineered to express or overexpress an acetyl-CoA carboxylase and an acetoacetyl-CoA synthase and further expresses or overexpresses one or more enzymes selected from the group consisting of (a) acetoacetyl-CoA reductase, (b) enoyl-CoA hydratase, (c) trans-2-enoyl-CoA reductase, and (d) butyraldehyde dehydrogenase and 1,3-propanediol dehydrogenase.

16. The recombinant microorganism of claim 8, wherein the microorganism is a photoautotrophic or photoheterotrophic organism and wherein is engineered to express or overexpress an acetyl-CoA carboxylase and an acetoacetyl-CoA synthase and one or more enzymes selected from the group consisting of (a) hydroxybutyryl CoA dehydrogenase, (b) crotonase, (c) crotonyl-CoA reductase, and (d) an alcohol/aldehyde dehydrogenase.

17. The recombinant microorganism of claim 8, wherein the microorganism comprises a photoautotrophic or photoheterotrophic organism that is engineered to express or overexpress an acetyl-CoA carboxylase and an acetoacetyl-CoA synthase and one or more enzymes selected from the group consisting of (a) hydroxybutyryl CoA dehydrogenase, (b) crotonase, (c) trans-2-enoyl-CoA reductase, and (d) an alcohol/aldehyde dehydrogenase.

18. The recombinant microorganism of claim 8, wherein the microorganism comprises a photoautotrophic or photoheterotrophic organism that is engineered to express or overexpress an acetyl-CoA carboxylase and an acetoacetyl-CoA synthase and one or more enzymes selected from the group consisting of (a) hydroxybutyryl CoA dehydrogenase, (b) crotonase, (c) trans-2-enoyl-CoA reductase, and (d) butyraldehyde dehydrogenase and 1,3-propanediol dehydrogenase.

19. The recombinant microorganism of claim 1, wherein the microorganism comprises a photoautotrophic or photoheterotrophic organism and includes the expression of at least one heterologous, or the over expression of at least one endogenous, target enzyme from the group consisting of an enzyme that converts (i) acetyl-CoA to malonyl-CoA, (ii) malonyl-CoA to Acetoacetyl-CoA, (iii) acetoacetyl-CoA to (R)- or (S)-3-hydroxybutyryl-CoA, (iv) (R)- or (S)-3-hydroxybutyryl-CoA to crotonyl-CoA, (v) crotonyl-CoA to butyryl-CoA, (vi) butyryl-CoA to butyraldehyde and (vi) butyraldehyde to 1-butanol.

20. The recombinant microorganism of claim 1, wherein the microorganism comprises a reduction, disruption or knockout of at least one gene encoding an enzyme that competes with a metabolite necessary for the production of a desired higher alcohol product or which produces an unwanted product.

21. The recombinant microorganism of claim 20, wherein the microorganism comprises a photoautotrophic or photoheterotrophic organism that is engineered to disrupt, delete or knockout one or more genes encoding a polypeptide or protein selected from the group consisting of: (i) an enzyme that catalyzes the NADH-dependent conversion of pyruvate to D-lactate (e.g., IdhA); (ii) an enzyme that promotes catalysis of fumarate and succinate interconversion (e.g., frdBC); (iii) an oxygen transcription regulator; and (iv) an enzyme that catalyzes the conversion of acetyl-coA to acetyl-phosphate (e.g., pta).

22. The recombinant microorganism of claim 21, comprises a disruption, deletion or knockout of a combination of an alcohol/acetoaldehyde dehydrogenase and one or more of (i)-(iv).

23. The recombinant microorganism of claim 1, wherein the microorganism is engineered to express one or more subunits of acetyl-coA carboxylase (AccABCD) that converts acetyl-CoA to malonyl-CoA.

24. The recombinant microorganism of claim 1, wherein the microorganism is engineered to express of over express one or more genes selected from the group consisting of nphT7, phaB, phaJ, ter, bldh, and yqhD, and wherein the microorganism produces 1-butanol.

25. The recombinant microorganism of claim 24, further comprising expressing or over expressing AccABCD.

26. The recombinant microorganism of claim 25, wherein the microorganism expresses a polypeptide having at least 90-100% identity to SEQ ID NO:2 (AccABCD).

27. The recombinant microorganism of claim 24, wherein the microorganism expresses a polypeptide having at least 90-100% identity to SEQ ID NO:18 (nphT7).

28. The recombinant microorganism of claim 24, wherein the microorganism expresses a polypeptide having at least 90-100% identity to SEQ ID NO:30 (phaB).

29. The recombinant microorganism of claim 24, wherein the microorganism expresses a polypeptide having at least 90-100% identity to SEQ ID N0:28 (phaJ).

30. The recombinant microorganism of claim 24, wherein the microorganism expresses a polypeptide having at least 90-100% identity to SEQ ID NO:23, 24, 25, or 26 (ter).

31. The recombinant microorganism of claim 24, wherein the microorganism expresses a polypeptide having at least 90-100% identity to SEQ ID NO:34 (Bldh).

32. The recombinant microorganism of claim 24, wherein the microorganism expresses a polypeptide having at least 90-100% identity to SEQ ID N0:32 (yqhD).

33. The recombinant microorganism of claim 1, wherein the microorganism comprises an expression profile selected from the group consisting of:
(a) AccABCD, nphT7, PhaB, PhaJ, Ter, BIdH, and YqhD;
(b) nphT7, PhaB, PhaJ, Ter, BIdH, and YqhD;
(c) AccABCD, nphT7, PhaB, PhaJ, Ter, and AdhE2;
(d) nphT7, PhaB, PhaJ, Ter, and AdhE2;
(e) AccABCD, nphT7, PhaB, PhaJ, ccr, BIdH, and YqhD;
(f) nphT7, PhaB, PhaJ, ccr, BIdH, and YqhD;
(g) AccABCD, nphT7, PhaB, PhaJ, ccr, and AdhE2;
(h) nphT7, PhaB, PhaJ, ccr, and AdhE2;
(i) AccABCD, nphT7, hbd, crt, Ter, BIdH, and YqhD;
(j) nphT7, hbd, crt, Ter, BIdH, and YqhD;
(k) AccABCD, nphT7, hbd, crt, Ter, and AdhE2; and
(l) nphT7, hbd, crt, Ter, and AdhE2.

34. A method for producing an alcohol, the method comprising:
a) providing a recombinant photoautotroph or photoheterotrophic microorganism of claim 1;
b) culturing the microorganism(s) of (a) in the presence of CO₂ under conditions suitable for the conversion of the substrate to an alcohol; and
c) purifying the alcohol.


SEQUENCE LISTING (35 sequences)

SEQ ID NO: 1 - DNA, 951 bp, Pseudomonas aeruginosa
  CDS (1)..(951)
atgaacccgaactttcttgatttcgaacagccgatcgccgacctgcaagccaagatcgaagagctgcgcctggtgggcaacgacaatgcgctgaacatcagcgacgaaatctcgcgtctgcaggacaagagcaaggcgctcaccgaaaacatcttcggcaatctgtccagttggcagatcgcccagctcgcgcgccatcccaagcgtccctataccctcgactacatcggctacctgttcagcgatttcgaggaactgcacggcgaccggcatttcgccgacgacccggcgatcgtcggcggcgttgcccgcctcgacggttccccggtgatggtcatcggccaccagaagggccgcgaagtccgtgagaaggtccggcgcaacttcggcatgccgcgtccggaaggctatcgcaaggcctgccgcctgatggaaatggccgaacgcttcaagatgccgatcctcaccttcatcgacacgcccggcgcctacccggggatcgatgccgaggaacgcggccagagcgaggcgatcgcctggaacctgcgggtgatggcgcgactgaagacgccgatcatcgccaccgtgatcggcgagggcggttccggcggcgcgctggccatcggtgtctgcgaccagttgaacatgctgcaatactccacctattcggtgatctcgccggaaggctgcgcctccatcctctggaagaccgccgagaaggcgccggaagccgccgaggccatgggcatcaccgccgagcgcctgaaaggcctgggcatcgtcgacaaggtcatcgacgaaccgctgggcggcgcccatcgcgatccggcgagcatggccgaatcgatccgtggcgaactgctggcgcaactgaagatgctccagggcctggaaatgggtgagttgctggagcgtcgttacgaccgcctgatgagctacggcgcgccgtaa

SEQ ID NO: 2 - protein, 316 aa, Pseudomonas aeruginosa
MNPNFLDFEQPIADLQAKIEELRLVGNDNALNISDEISRLQDKSKALTENIFGNLSSWQIAQLARHPKRPYTLDYIGYLFSDFEELHGDRHFADDPAIVGGVARLDGSPVMVIGHQKGREVREKVRRNFGMPRPEGYRKACRLMEMAERFKMPILTFIDTPGAYPGIDAEERGQSEAIAWNLRVMARLKTPIIATVIGEGGSGGALAIGVCDQLNMLQYSTYSVISPEGCASILWKTAEKAPEAAEAMGITAERLKGLGIVDKVIDEPLGGAHRDPASMAESIRGELLAQLKMLQGLEMGELLERRYDRLMSYGAP

SEQ ID NO: 3 - DNA, 1344 bp, Streptomyces coelicolor
  CDS (1)..(1344)
gtgaccgtgaaggacatcctggacgcgatccagtcgcccgactccacgccggccgacatcgccgcactgccgctccccgagtcgtaccgcgcgatcaccgtgcacaaggacgagaccgagatgttcgcgggcctcgagacccgcgacaaggacccccgcaagtcgatccacctggacgacgtgccggtgcccgagctgggccccggcgaggccctggtggccgtcatggcctcctcggtcaactacaactcggtgtggacctcgatcttcgagccgctgtccaccttcgggttcctggagcgctacggccgggtcagcgacctcgccaagcggcacgacctgccgtaccacgtcatcggctccgacctcgccggtgtcgtcctgcgcaccggtccgggcgtcaacgcctggcaggcgggcgacgaggtcgtcgcgcactgcctctccgtcgagctggagtcctccgacggccacaacgacacgatgctcgaccccgagcagcgcatctggggcttcgagaccaacttcggcggcctcgcggagatcgcgctggtcaagtccaaccagctgatgccgaagccggaccacctgagctgggaggaggccgccgctcccggcctggtcaactccaccgcgtaccgccagctcgtctcccgcaacggcgccggcatgaagcagggcgacaacgtgctcatctggggcgcgagcggcggactcggctcgtacgccacccagttcgccctcgccggcggcgccaacccgatctgcgtcgtctcctcgccgcagaaggcggagatctgccgcgcgatgggcgccgaggcgatcatcgaccgcaacgccgagggctaccggttctggaaggacgagaacacccaggacccgaaggagtggaagcgcttcggcaagcgcatccgcgaactgaccggcggcgaggacatcgacatcgtcttcgagcaccccggccgcgagaccttcggcgcctccgtcttcgtcacccgcaagggcggcaccatcaccacctgcgcctcgacctcgggctacatgcacgagtacgacaaccgctacctgtggatgtccctgaagcgcatcatcggctcgcacttcgccaactaccgcgaggcctgggaggccaaccgcctcatcgccaagggcaggatccaccccacgctctccaaggtgtactccctcgaggacaccggccaggccgcctacgacgtccaccgcaacctccaccagggcaaggtcggcgtgctgtgcctggcgcccgaggagggcctgggcgtgcgcgaccgggagaagcgcgcgcagcacctcgacgccatcaaccgcttccggaacatctga

SEQ ID NO: 4 - protein, 447 aa, Streptomyces coelicolor
VTVKDILDAIQSPDSTPADIAALPLPESYRAITVHKDETEMFAGLETRDKDPRKSIHLDDVPVPELGPGEALVAVMASSVNYNSVWTSIFEPLSTFGFLERYGRVSDLAKRHDLPYHVIGSDLAGVVLRTGPGVNAWQAGDEVVAHCLSVELESSDGHNDTMLDPEQRIWGFETNFGGLAEIALVKSNQLMPKPDHLSWEEAAAPGLVNSTAYRQLVSRNGAGMKQGDNVLIWGASGGLGSYATQFALAGGANPICVVSSPQKAEICRAMGAEAIIDRNAEGYRFWKDENTQDPKEWKRFGKRIRELTGGEDIDIVFEHPGRETFGASVFVTRKGGTITTCASTSGYMHEYDNRYLWMSLKRIIGSHFANYREAWEANRLIAKGRIHPTLSKVYSLEDTGQAAYDVHRNLHQGKVGVLCLAPEEGLGVRDREKRAQHLDAINRFRNI

SEQ ID NO: 5 - DNA, 1206 bp, Pseudomonas syringae
  CDS (1)..(1206)
atgaatcaagcactgactgaaaccatgcaggcctttctgatccgccccgagcgctatggcgaaccgcagcaggccatccagctcgaacaggtccagatccccaccctgggtccgcatcaggtcctcatcgaagtgatggcagccggactcaactacaacaacgtctgggccgcccagggtaagccggtggacatcatcgccgcgcggcgcaagcggaaccgtgacgccgaacccttccacatcggaggctcggaagcctccggttacgtgaaagccgtgggcgacgctgtcacccacgtcaaggtgggcgataccgtggtggtgtcctgctcggtctacgacgccacggccatcgaatcgcgcgtcgcccccgaccccatgttctgcagcaaccaggaaatctacggctacgagaccagctacggctccttcgccgaatacaccctcgtcgaagactaccaatgcttcccaaaaccaaagttcctgagctgggaggaaagtgccaccctgatgctcaatggtccgaccgcctacaagcagctcacgcattgggcacccaataccgtcaagcctggagacgcagtcctgatctggggcgcggcaggtggcctgggctctatgtctatccagttgacccgcgcgctcggggggctgccggtggccgtggtgtccagtccagacaggggccgctacgcctgcgaactcggcgccgtggggtacttgctcagaaccgactatccgcacctgggacgtctgccggacttgaactccgacgctcacagcgcctggaccaaaagcttcgcgagtttccgtcgcgacttcttcatgacgctggggaaaaaggagctgcccaaagtggtgatcgagcactccggccaagccaccttccccacctcgctgcagatctgcgaccgctccggcatggtggtcatcgtgggtggcacgtccggctacaactgcgacttcgatgtccgccacctgtggatgcaccagaagcgcatccagggctcccactacgccaacatccgcgagtgccaggaattcctgcaactagtcgaacaacgccgggtagtgccgaccctgaacaccctctatcgcttcgaggagacacctagggcgcatcaggcgctactgagtggagaagtcgtaggcaatgccgccgtgctggtcaaggccgagcgacccggcctaggggtcggttgttga

SEQ ID NO: 6 - protein, 401 aa, Pseudomonas syringae
MNQALTETMQAFLIRPERYGEPQQAIQLEQVQIPTLGPHQVLIEVMAAGLNYNNVWAAQGKPVDIIAARRKRNRDAEPFHIGGSEASGYVKAVGDAVTHVKVGDTVVVSCSVYDATAIESRVAPDPMFCSNQEIYGYETSYGSFAEYTLVEDYQCFPKPKFLSWEESATLMLNGPTAYKQLTHWAPNTVKPGDAVLIWGAAGGLGSMSIQLTRALGGLPVAVVSSPDRGRYACELGAVGYLLRTDYPHLGRLPDLNSDAHSAWTKSFASFRRDFFMTLGKKELPKVVIEHSGQATFPTSLQICDRSGMVVIVGGTSGYNCDFDVRHLWMHQKRIQGSHYANIRECQEFLQLVEQRRVVPTLNTLYRFEETPRAHQALLSGEVVGNAAVLVKAERPGLGVGC

SEQ ID NO: 7 - DNA, 1293 bp, Rhodobacter sphaerides
  CDS (1)..(1293)
atggccctcgacgtgcagagcgatatcgtcgcctacgacgcgcccaagaaggacctctacgagatcggcgagatgccgcctctcggccatgtgccgaaggagatgtatgcttgggccatccggcgcgagcgtcatggcgagccggatcaggccatgcagatcgaggtggtcgagacgccctcgatcgacagccacgaggtgctcgttctcgtgatggcggcgggcgtgaactacaacggcatctgggccggcctcggcgtgcccgtctcgccgttcgacggtcacaagcagccctatcacatcgcgggctccgacgcgtcgggcatcgtctgggcggtgggcgacaaggtcaagcgctggaaggtgggcgacgaggtcgtgatccactgcaaccaggacgacggcgacgacgaggaatgcaacggcggcgacccgatgttctcgcccacccagcggatctggggctacgagacgccggacggctccttcgcccagttcacccgcgtgcaggcgcagcagctgatgaagcgtccgaagcacctgacctgggaagaggcggcctgctacacgctgaccctcgccaccgcctaccggatgctcttcggccacaagccgcacgacctgaagccggggcagaacgtgctggtctggggcgcctcgggcggcctcggctcctacgcgatccagctcatcaacacggcgggcgccaatgccatcggcgtcatctcagaggaagacaagcgcgacttcgtcatggggctgggcgccaagggcgtcatcaaccgcaaggacttcaagtgctggggccagctgcccaaggtgaactcgcccgaatataacgagtggctgaaggaggcgcgcaagttcggcaaggccatctgggacatcaccggcaagggcatcaacgtcgacatggtgttcgaacatccgggcgaggcgaccttcccggtctcgtcgctggtggtgaagaagggcggcatggtcgtgatctgcgcgggcaccaccggcttcaactgcaccttcgacgtccgctacatgtggatgcaccagaagcgcctgcagggcagccatttcgccaacctcaagcaggcctccgcggccaaccagctgatgatcgagcgccgcctcgatccctgcatgtccgaggtcttcccctgggccgagatcccggctgcccatacgaagatgtataagaaccagcacaagcccggcaacatggcggtgctggtgcaggccccgcgcacggggttgcgcaccttcgccgacgtgctcgaggccggccgcaaggcctga

SEQ ID NO: 8 - protein, 430 aa, Rhodobacter sphaerides
MALDVQSDIVAYDAPKKDLYEIGEMPPLGHVPKEMYAWAIRRERHGEPDQAMQIEVVETPSIDSHEVLVLVMAAGVNYNGIWAGLGVPVSPFDGHKQPYHIAGSDASGIVWAVGDKVKRWKVGDEVVIHCNQDDGDDEECNGGDPMFSPTQRIWGYETPDGSFAQFTRVQAQQLMKRPKHLTWEEAACYTLTLATAYRMLFGHKPHDLKPGQNVLVWGASGGLGSYAIQLINTAGANAIGVISEEDKRDFVMGLGAKGVINRKDFKCWGQLPKVNSPEYNEWLKEARKFGKAIWDITGKGINVDMVFEHPGEATFPVSSLVVKKGGMVVICAGTTGFNCTFDVRYMWMHQKRLQGSHFANLKQASAANQLMIERRLDPCMSEVFPWAEIPAAHTKMYKNQHKPGNMAVLVQAPRTGLRTFADVLEAGRKA

SEQ ID NO: 9 - DNA, 1284 bp, Rhodospirillum rubrum
  CDS (1)..(1284)
atgaccacgtcggcggaagtcatagaactcaatcccggcactggccggaaggatctttacgaactcggtgaaattccgccgctcggccacgttcccaagtctatgtacgcctgggtcatccgccgggatcgccatggcgaacccgagaagtctttccaggttgaagtcgttgaaacgccaactcttgacagccacgacgtcttggtgatggtgatggcggccggcgtcaactacaacgggatctgggccggattgggccagccgatcagcgttttcgactcgcataaggccgcttatcacatcgccggttcggatgcggcgggcatcgtctgggccgtcggcgccaaggtcaagcgctggaaggtcggcgacgaggtggtcgtccactgcaatcagaccgacggcgacgacgaggaatgcaatggtggcgatccgatgttctcgccgacccagcgcatctggggctatgagacccccgatggctccttcgcccagttcacccgcgtgcagtcccagcaggtgatggcccgtccgcgccatctgacctgggaggaaagtgccagctacgtgctggttctggccaccgcctatcgcatgctgttcggccaccgcccccatgtgctgcgcccgggtcacaacgtgctgatctggggcgcctcgggcggcctgggatcgatggcgatccagctgtgcgccacggcgggcgccaatgccatcggcgtcatctccgatgagaccaagcgcgatttcgtcatgagcctgggcgccaagggcgtgatcaaccgcaaggatttcaattgctggggccaattgcccacggtcaatggcgagggcttcgacgcctatatgaaagaggtgcgcaagttcggcaaggcgatctgggacatcaccggcaagggcaacgacgttgatttcgtgttcgaacatccgggcgagcagaccttcccggtctcgtgcaatgtggtcaagcgcggtggcatggtggtgttttgcgccggcaccaccggcttcaacctgaccttcgacgcccgctttgtgtggatgcgccagaagcgcattcagggcagccacttcgccaatctgctccaggcctcgcaagccaaccagttggtcatcgagcggcggatcgatccgtgcatgagcgaagtgttttcctgggacgatattcccaaggcccacaccaagatgtggaagaatcagcataagccggggaatatggcggtgctggtccaggcccatcgcccgggccgccgcaccttggaggattgccgagaggaagggtga

SEQ ID NO: 10 - protein, 427 aa, Rhodospirillum rubrum
MTTSAEVIELNPGTGRKDLYELGEIPPLGHVPKSMYAWVIRRDRHGEPEKSFQVEVVETPTLDSHDVLVMVMAAGVNYNGIWAGLGQPISVFDSHKAAYHIAGSDAAGIVWAVGAKVKRWKVGDEVVVHCNQTDGDDEECNGGDPMFSPTQRIWGYETPDGSFAQFTRVQSQQVMARPRHLTWEESASYVLVLATAYRMLFGHRPHVLRPGHNVLIWGASGGLGSMAIQLCATAGANAIGVISDETKRDFVMSLGAKGVINRKDFNCWGQLPTVNGEGFDAYMKEVRKFGKAIWDITGKGNDVDFVFEHPGEQTFPVSCNVVKRGGMVVFCAGTTGFNLTFDARFVWMRQKRIQGSHFANLLQASQANQLVIERRIDPCMSEVFSWDDIPKAHTKMWKNQHKPGNMAVLVQAHRPGRRTLEDCREEG

SEQ ID NO: 11 - DNA, 1338 bp, Streptomyces avermitilis
  CDS (1)..(1338)
gtgaaggaaatcctggacgcgattcagtcccagacggccacgtctgccgacttcgccgcactgccgctccccgactcgtaccgcgcgatcaccgtgcacaaggacgagacggagatgttcgccgggctcagcacccgcgacaaggacccccgcaagtcgatccacctggacgacgtgccggtgccggagctcggccccggcgaggccctggtggccgtcatggcgtcctccgtgaactacaactcggtctggacgtcgatcttcgagccggtgtcgaccttcaacttcctggagcgctacgggcggctcagcgatctcagcaagcgccacgacctgccgtaccacatcatcggttctgacctcgcgggcgtcgtcctgcgcaccggcccgggagtcaactcctggaagcccggcgacgaggtcgtcgcgcactgtctctcggtcgagctggagtcgtccgacggccacaacgacacgatgctcgaccccgagcagcgcatctggggcttcgagaccaacttcggcgggctcgccgagatcgcgctcgtcaagtccaaccagctgatgccgaagccggaccacctcagctgggaggaggccgccgctccgggcctggtgaactcgaccgcgtaccggcagctcgtctcccgcaacggcgccggcatgaagcagggcgacaacgtcctcatctggggcgcgagcggtggactgggctcgtacgccacgcagttcgcgctcgccggcggcgccaacccgatctgcgtcgtctccagcgagcagaaggcggacatctgccgctcgatgggcgccgaggcgatcatcgaccgcaacgccgagggctacaagttctggaaggacgagaccacccaggacccgaaggagtggaagcgcttcggcaagcgcatccgcgagttcaccggcggcgaggacatcgacatcgtcttcgagcaccccggccgcgagaccttcggcgcctcggtctacgtcacccgcaagggcggcaccatcaccacctgcgcctcgacctcgggctacatgcacgagtacgacaaccgctacctgtggatgtcgctgaagcggatcatcggctcgcacttcgcgaactaccgcgaggcctgggaggccaaccgcctcgtcgccaagggcaagatccaccccacgctctccaaggtctactccctggaggacaccgggcaggccgcctacgacgtgcaccgcaacctccaccagggcaaggtcggcgtgctcgccctcgcgccccgcgagggcctgggcgtgcgcgacgaggagaagcgcgcgcagcacatcgacgccatcaaccgcttccggaacatctga

SEQ ID NO: 12 - protein, 445 aa, Streptomyces avermitilis
VKEILDAIQSQTATSADFAALPLPDSYRAITVHKDETEMFAGLSTRDKDPRKSIHLDDVPVPELGPGEALVAVMASSVNYNSVWTSIFEPVSTFNFLERYGRLSDLSKRHDLPYHIIGSDLAGVVLRTGPGVNSWKPGDEVVAHCLSVELESSDGHNDTMLDPEQRIWGFETNFGGLAEIALVKSNQLMPKPDHLSWEEAAAPGLVNSTAYRQLVSRNGAGMKQGDNVLIWGASGGLGSYATQFALAGGANPICVVSSEQKADICRSMGAEAIIDRNAEGYKFWKDETTQDPKEWKRFGKRIREFTGGEDIDIVFEHPGRETFGASVYVTRKGGTITTCASTSGYMHEYDNRYLWMSLKRIIGSHFANYREAWEANRLVAKGKIHPTLSKVYSLEDTGQAAYDVHRNLHQGKVGVLALAPREGLGVRDEEKRAQHIDAINRFRNI

SEQ ID NO: 13 - DNA, 1287 bp, Silicibacter pomeroyi
  CDS (1)..(1287)
atggctttggacaccgacagcggtatcgcgtcctacgcggcgcccgagaaagacctctatgagatgggtgaaatccccccgatgggattcgtgcccaagaagatgtatgcgtgggcgatccgcaaagagcgccacggtgatcccgataccgcgatgcaggtcgaagtggttgacgtgccgacgctcgacagccacgaggtgctggttctggtgatggccgctggcgtcaactacaatggcgtctgggcctccaaaggtgttccgatttcccccttcgatggccacggacagccctatcacatcgccggttccgatgcttcgggtatcgtctgggccgtgggggacaaggtcaagcgctggaaggtcggcgacgaggtcgtgatccactgcaatcaggatgatggtgacgacgagcactgcaatggcggtgacccgatgtattcgcccagtcagcggatctggggttacgagacgccggacggatcctttgctcagttcaccaatgtgcaggcgcagcagctgatgccgcggcccaagcacctgacctgggaagaagcggcatgttacacgctgacgctggcgaccgcctaccggatgctgtttggccatgagccgcatgatctcaagcccggtcagaacgttctggtctggggtgcgtccggtggtctggggtcctatgcgatccagcttatcaatacggcgggtgcgaacgcgattggcgtcatctcggatgaaagcaagcgccagtttgtcatggaccttggcgcaaagggtgtcatcaaccgcaaggatttcaactgctggggtcaactgcccacggtgaacacccccgaatatgccgagtggttcaaggaagcccgcaagttcggcaaggcgatctgggacattaccggcaagggcgtgaacgtggacatggtcttcgagcaccccggcgagagcacgttcccggtctcgaccttcgtggtgaagaagggcggtatggttgtgatctgcgcgggcaccagcggctacaacctgacctttgacgtgcgctatatgtggatgcaccagaagcgccttcagggcagccacttcgcccatctcaagcaggcaatggccgcgaaccagctgatggtcgagcgccggctcgacccgtgcatgtccgaggtgttcacctgggccgatctgcccgaggcgcatatgaagatgatgcgcaacgagcacaagccgggcaacatgtcggtgctggtgcaatcgccccgcaccgggctgcgcaccctcgaagaggttctggacgcccgcggttaa

SEQ ID NO: 14 - protein, 428 aa, Silicibacter pomeroyi
MALDTDSGIASYAAPEKDLYEMGEIPPMGFVPKKMYAWAIRKERHGDPDTAMQVEVVDVPTLDSHEVLVLVMAAGVNYNGVWASKGVPISPFDGHGQPYHIAGSDASGIVWAVGDKVKRWKVGDEVVIHCNQDDGDDEHCNGGDPMYSPSQRIWGYETPDGSFAQFTNVQAQQLMPRPKHLTWEEAACYTLTLATAYRMLFGHEPHDLKPGQNVLVWGASGGLGSYAIQLINTAGANAIGVISDESKRQFVMDLGAKGVINRKDFNCWGQLPTVNTPEYAEWFKEARKFGKAIWDITGKGVNVDMVFEHPGESTFPVSTFVVKKGGMVVICAGTSGYNLTFDVRYMWMHQKRLQGSHFAHLKQAMAANQLMVERRLDPCMSEVFTWADLPEAHMKMMRNEHKPGNMSVLVQSPRTGLRTLEEVLDARG

SEQ ID NO: 15 - DNA, 1284 bp, Xanthobacter autotrophicus
  CDS (1)..(1284)
atggcccagacggcagccgccaacgcgaacgagggaccggtgaaggacctttatgagctgggcgaggttccccccctcggtcacgtccccgccaagatgtacgcctgggccatccgccgcgagcgccatgggccgccggaagagtcgttccagctggaagtggtgcccacctgggagctgggcgagaacgacgtgctggtctacgtcatggccgccggcgtcaactacaacggcatctgggcgggcctcggccagccgatctcgccgttcgacgtgcacaaggcgcccttccacatcgccggctccgatgcctcgggtatcgtctgggcggtgggctccaaggtgaagcgctggaaggtgggcgacgaggtggtcgtgcactgtaaccaggacgacggcgacgacgaggagtgcaacggcggcgaccccatgttctccccgtcccagcgcatctggggctatgagacgccggacggctcgttcgcccagttctgccgggtgcaggcgcgccagctgatgccgcgccccaagcacctgacctgggaagagagcgcctgctacaccctcaccatggccaccgcctaccgcatgctgttcggccatccgccgcacacggtgaagccgggcgactacgtgctggtgtggggcgcctcgggcggcctcggcgtgttcggcgtgcagctcgccgccgcctccggcgcccatgtgatcggcgtgatctccgacgagaccaagcgcgactatgtcctcggcctcggcgccaagggcgtgatcaaccgcaaggatttcaagtgctggggccagctgcccaaggtcaactcgccggaatacaatgagtggaccaaggaagcccgcaagttcggcaaggccatttgggacatcagcggcaagcgcgacgtggacatcgtgttcgagcatcctggcgagcagaccttcccggtctcgaccctcgtgggcaagcgcggcggcatgatcgtgttctgcgccggcaccaccggcttcaacatcaccttcgacgcccgctacgtgtggatgcgccagaagcgcatccagggctcccacttcgctcacctcaagcaggcctccgccgccaatcagttcatcatcgaccggcgcgtggacccctgcatgtcggaagtgtttccgtgggaccgcatccccgaggcgcacaccaagatgtggaagaaccagcacgcccctggcaacatggcggtgctggtcaacaccccccgcaccggcctgcgtaccctcgaggacgtgatcgaggccggcgcgaagaagtga

SEQ ID NO: 16 - protein, 427 aa, Xanthobacter autotrophicus
MAQTAAANANEGPVKDLYELGEVPPLGHVPAKMYAWAIRRERHGPPEESFQLEVVPTWELGENDVLVYVMAAGVNYNGIWAGLGQPISPFDVHKAPFHIAGSDASGIVWAVGSKVKRWKVGDEVVVHCNQDDGDDEECNGGDPMFSPSQRIWGYETPDGSFAQFCRVQARQLMPRPKHLTWEESACYTLTMATAYRMLFGHPPHTVKPGDYVLVWGASGGLGVFGVQLAAASGAHVIGVISDETKRDYVLGLGAKGVINRKDFKCWGQLPKVNSPEYNEWTKEARKFGKAIWDISGKRDVDIVFEHPGEQTFPVSTLVGKRGGMIVFCAGTTGFNITFDARYVWMRQKRIQGSHFAHLKQASAANQFIIDRRVDPCMSEVFPWDRIPEAHTKMWKNQHAPGNMAVLVNTPRTGLRTLEDVIEAGAKK

SEQ ID NO: 17 - DNA, 2137 bp, Streptomyces sp. CL190
  CDS (647)..(1636)
cctgcaggccgtcgagggcgcctggaaggactacgcggagcaggacggccggtcgctggaggagttcgcggcgttcgtctaccaccagccgttcacgaagatggcctacaaggcgcaccgccacctgctgaacttcaacggctacgacaccgacaaggacgccatcgagggcgccctcggccagacgacggcgtacaacaacgtcatcggcaacagctacaccgcgtcggtgtacctgggcctggccgccctgctcgaccaggcggacgacctgacgggccgttccatcggcttcctgagctacggctcgggcagcgtcgccgagttcttctcgggcaccgtcgtcgccgggtaccgcgagcgtctgcgcaccgaggcgaaccaggaggcgatcgcccggcgcaagagcgtcgactacgccacctaccgcgagctgcacgagtacacgctcccgtccgacggcggcgaccacgccaccccggtgcagaccaccggccccttccggctggccgggatcaacgaccacaagcgcatctacgaggcgcgctagcgacacccctcggcaacggggtgcgccactgttcggcgcaccccgtgccgggctttcgcacagctattcacgaccatttgaggggcgggcagccgcatgaccgacgtccgattccgcattatcggtacgggtgcctacgtaccggaacggatcgtctccaacgatgaagtcggcgcgccggccggggtggacgacgactggatcacccgcaagaccggtatccggcagcgtcgctgggccgccgacgaccaggccacctcggacctggccacggccgcggggcgggcagcgctgaaagcggcgggcatcacgcccgagcagctgaccgtgatcgcggtcgccacctccacgccggaccggccgcagccgcccacggcggcctatgtccagcaccacctcggtgcgaccggcactgcggcgttcgacgtcaacgcggtctgctccggcaccgtgttcgcgctgtcctcggtggcgggcaccctcgtgtaccggggcggttacgcgctggtcatcggcgcggacctgtactcgcgcatcctcaacccggccgaccgcaagacggtcgtgctgttcggggacggcgccggcgcaatggtcctcgggccgacctcgaccggcacgggccccatcgtccggcgcgtcgccctgcacaccttcggcggcctcaccgacctgatccgtgtgcccgcgggcggcagccgccagccgctggacacggatggcctcgacgcgggactgcagtacttcgcgatggacgggcgtgaggtgcgccgcttcgtcacggagcacctgccgcagctgatcaagggcttcctgcacgaggccggggtcgacgccgccgacatcagccacttcgtgccgcatcaggccaacggtgtcatgctcgacgaggtcttcggcgagctgcatctgccgcgggcgaccatgcaccggacggtcgagacctacggcaacacgggagcggcctccatcccgatcaccatggacgcggccgtgcgcgccggttccttccggccgggcgagctggtcctgctggccgggttcggcggcggcatggccgcgagcttcgccctgatcgagtggtagtcgcccgtaccaccacagcggtccggcgccacctgttccctgcgccgggccgccctcggggcctttaggccccacaccgccccagccgacggattcagtcgcggcagtacctcagatgtccgctgcgacggcgtcccggagagcccgggcgagatcgcgggcccccttctgctcgtccccggcccctcccgcgagcaccacccgcggcggacggccgccgtcctccgcgatacgccgggcgaggtcgcaggcgagcacgccggacccggagaagccccccagcaccagcgaccggccgactccgtgcgcggccagggcaggctgcgcgccgtcgacgtcggtgagcagcaccaggagctcctgcggcccggcgtagaggtcggccagccggtcgtagcaggtcgcgggcgcgcccggcggcgggatcagacagatcgtgcccgcccgctcgtgcctcgccgcccgcagcgtgaccagcggaatgtcccgcccagctccgga

SEQ ID NO: 18 - protein, 329 aa, Streptomyces sp. CL190
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 19 - DNA, 849 bp, Clostridium acetobutylicum
  CDS (1)..(849)
atgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataa

SEQ ID NO: 20 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 21 - DNA, 786 bp, Clostridium acetobutylicum
  CDS (1)..(786)
atggaactaaacaatgtcatccttgaaaaggaaggtaaagttgctgtagttaccattaacagacctaaagcattaaatgcgttaaatagtgatacactaaaagaaatggattatgttataggtgaaattgaaaatgatagcgaagtacttgcagtaattttaactggagcaggagaaaaatcatttgtagcaggagcagatatttctgagatgaaggaaatgaataccattgaaggtagaaaattcgggatacttggaaataaagtgtttagaagattagaacttcttgaaaagcctgtaatagcagctgttaatggttttgctttaggaggcggatgcgaaatagctatgtcttgtgatataagaatagcttcaagcaacgcaagatttggtcaaccagaagtaggtctcggaataacacctggttttggtggtacacaaagactttcaagattagttggaatgggcatggcaaagcagcttatatttactgcacaaaatataaaggcagatgaagcattaagaatcggacttgtaaataaggtagtagaacctagtgaattaatgaatacagcaaaagaaattgcaaacaaaattgtgagcaatgctccagtagctgttaagttaagcaaacaggctattaatagaggaatgcagtgtgatattgatactgctttagcatttgaatcagaagcatttggagaatgcttttcaacagaggatcaaaaggatgcaatgacagctttcatagagaaaagaaaaattgaaggcttcaaaaatagatag

SEQ ID NO: 22 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 23 - protein, 398 aa, Treponema denticola
  misc_feature (398)..(398): Xaa can be any naturally occurring amino acid
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRIX

SEQ ID NO: 24 - protein, 399 aa, Treponema vincentii
  misc_feature (399)..(399): Xaa can be any naturally occurring amino acid
MSMKPMLRSNICLNAHPQGCKKAVEDQIAYTRKRAASHPAGTATPKNVLVIGCSGGYGLASRITAAFGYGAATIGVSYEKAGSEKKWGTPGWYNNLAVDAAAKEAGLISVTINGDAFSDAIKAQVIDEAKKLNIKFDLIVYSVASSVRTDPDTGVTYRSALKPFGKPFTGKTLDPFTGALTEITAEPATDEEAAATVKVMGGEDWQRWIEKLGAADVLAQGCITVAYSYIGPEATQALYRKGTIGKAKEHLEATAHALNAKLAALKGKAFVSVNKGLVTRASAVIPVIPLYLASLFKVMKEKGTHEGCIEQINRLFDSRLYTADGVIPTDNENRIRIDDWELDEGVQSAVAKIMATVTDETSCKLTDVDEYRHDFLAINGFDIAGIDYDAEIDRFDRIX

SEQ ID NO: 25 - protein, 406 aa, Fibrobacter succinogenes
  misc_feature (406)..(406): Xaa can be any naturally occurring amino acid
MIIKPLIRSNMCINAHPKGCAADVKHQIEFIKKKFTTRSIPADAPKTVLVLGCSTGYGLASRIVAAFGYKAATIGVSFEKEGSDGGIGESREKTGTPGWYNNMAFDKFAKEAGLDAVTFNGDAFSHEMRQNVIDTLKKMGRKVDLLVYSVASSVRVDPDNGTIYRSVLKPIDKVFTGATIDCLSGKISTISAEPATAEEAANTVKVMGGEDWALWVRKLKEAGVLAEGVKTVAYSYIGPKLSHAIYRDGTIGGAKKHLEATALELNKELQNDLHGEAYVSVNKGLVTRSSAVIPIIPMYISVLFKVMKEMGNHEGCIEQMERLMTERLYTGSKVPTDENHLIRIDDYELDPKVQAEVDKRMATVTQENLAEVGDLEGYRHDFLATNGFDIDGVDYEADVQTLTSIX

SEQ ID NO: 26 - protein, 397 aa, Flavobacterium johnsoniae
  misc_feature (397)..(397): Xaa can be any naturally occurring amino acid
MIIEPRMRGFICLTAHPAGCEQNVKNQIEYIKSKGAIAGAKKVLVIGASTGFGLASRITSAFGSDAATIGVFFEKPPVEGKTASPGWYNSAAFEKEAHKAGLYAKSINGDAFSNEIKRETLDLIKADLGQVDLVIYSLASPVRTNPNTGVTHRSVLKPIGQTFTNKTVDFHTGNVSEVSIAPANEEDIENTVAVMGGEDWAMWIDALKNENLLAEGATTIAYSYIGPELTEAVYRKGTIGRAKDHLEATAFTITDTLKSLGGKAYVSVNKALVTQASSAIPVIPLYISLLYKIMKEEGIHEGCIEQIQRLFQDRLYNGSEVPVDEKGRIRIDDWEMREDVQAKVAALWKEATTETLPSIGDLAGYRNDFLNLFGFEFAGVDYKADTNEVVNIESIKX

SEQ ID NO: 27 - DNA, 405 bp, Aeromonas caviae
  CDS (1)..(405)
atgagcgcacaatccctggaagtaggccagaaggcccgtctcagcaagcggttcggggcggcggaggtagccgccttcgccgcgctctcggaggacttcaaccccctgcacctggacccggccttcgccgccaccacggcgttcgagcggcccatagtccacggcatgctgctcgccagcctcttctccgggctgctgggccagcagttgccgggcaaggggagcatctatctgggtcaaagcctcagcttcaagctgccggtctttgtcggggacgaggtgacggccgaggtggaggtgaccgcccttcgcgaggacaagcccatcgccaccctgaccacccgcatcttcacccaaggcggcgccctcgccgtgacgggggaagccgtggtcaagctgccttaa

SEQ ID NO: 28 - protein, 134 aa, Aeromonas caviae
MSAQSLEVGQKARLSKRFGAAEVAAFAALSEDFNPLHLDPAFAATTAFERPIVHGMLLASLFSGLLGQQLPGKGSIYLGQSLSFKLPVFVGDEVTAEVEVTALREDKPIATLTTRIFTQGGALAVTGEAVVKLP

SEQ ID NO: 29 - DNA, 711 bp, Ralstonia eutropha H16
  CDS (1)..(711)
ttactgcatgtgctgcccgccattgatggcgatattggacccggtcacgtaggccgcttcctccgagcacaggtaggcaatcagcgctgcgacttcttccggcttgccgacgcggccgaccggaatctgcggcaggatcttggtctccatgatttccttgggcacggcgttgaccatcttggtggcaaggtagcccggagagacggtgttgacggtcacgcccttccttgccacttccagcgccagcgacttggtgaagccgtgcatcccggccttggcggcggcatagttggtctggccgaaggcgcccttggatgcgttgaccgatgagatgttgatgatgcgtccccagccgcgttcgaccatgccttcgcaaagcggcttggtgagattgaacaccgagtcgagattggtccgcatcacggcgtcccagttcggcttgtccatcttcttgaaggccatgtcgcgggtaatgccggcgttgttcaccaggatatccacgcggcctacgtcagccaggatctgcgccgcgcaagcctggcaggcatcgtagtcggatacatccacctcgtaggcacgtatctcgcggccgccggcggccatcgcggcaagccagtcctgggcggccgcattgcccggcgagtgcgtcaccaccaccgcatagcctgcgtcgtgcagcttgatgctgatggcttctcccagcccacccat

SEQ ID NO: 30 - protein, 109 aa, Ralstonia eutropha H16
LLHVLPAIDGDIGPGHVGRFLRAQVGNQRCDFFRLADAADRNLRQDLGLHDFLGHGVDHLGGKVARRDGVDGHALPCHFQRQRLGEAVHPGLGGGIVGLAEGALGCVDR

SEQ ID NO: 31 - protein, 127 aa, Ralstonia eutropha H16
DVDDASPAAFDHAFAKRLGEIEHRVEIGPHHGVPVRLVHLLEGHVAGNAGVVHQDIHAAYVSQDLRRASLAGIVVGYIHLVGTYLAAAGGHRGKPVLGGRIARRVRHHHRIACVVQLDADGFSQPTH

SEQ ID NO: 32 - DNA, 1164 bp, Escherichia coli
  CDS (1)..(1164)
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 33 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 34 - DNA, 1407 bp, Clostridium saccharoperbutylacetonicum N1-4(HMT)
  CDS (1)..(1407)
atgattaaagacacgctagtttctataacaaaagatttaaaattaaaaacaaatgttgaaaatgccaatctaaagaactacaaggatgattcttcatgtttcggagttttcgaaaatgttgaaaatgctataagcaatgccgtacacgcacaaaagatattatcccttcattatacaaaagaacaaagagaaaaaatcataactgagataagaaaggccgcattagaaaataaagagattctagctacaatgattcttgaagaaacacatatgggaagatatgaagataaaatattaaagcatgaattagtagctaaatacactcctgggacagaagatttaactactactgcttggtcaggagataacgggcttacagttgtagaaatgtctccatatggcgttataggtgcaataactccttctacgaatccaactgaaactgtaatatgtaatagtataggcatgatagctgctggaaatactgtggtatttaacggacatccaggcgctaaaaaatgtgttgcttttgctgtcgaaatgataaataaagctattatttcatgtggtggtcctgagaatttagtaacaactataaaaaatccaactatggactctctagatgcaattattaagcacccttcaataaaactactttgcggaactggagggccaggaatggtaaaaaccctcttaaattctggtaagaaagctataggtgctggtgctggaaatccaccagttattgtagatgatactgctgatatagaaaaggctggtaagagtatcattgaaggctgttcttttgataataatttaccttgtattgcagaaaaagaagtatttgtttttgagaacgttgcagatgatttaatatctaacatgctaaaaaataatgctgtaattataaatgaagatcaagtatcaaagttaatagatttagtattacaaaaaaataatgaaactcaagaatactctataaataagaaatgggtcggaaaagatgcaaaattattcttagatgaaatagatgttgagtctccttcaagtgttaaatgcataatctgcgaagtaagtgcaaggcatccatttgttatgacagaactcatgatgccaatattaccaattgtaagagttaaagatatagatgaagctattgaatatgcaaaaatagcagaacaaaatagaaaacatagtgcctatatttattcaaaaaatatagacaacctaaataggtttgaaagagaaatcgatactactatctttgtaaagaatgctaaatcttttgccggtgttggttatgaagcagaaggctttacaactttcactattgctggatccactggtgaaggaataacttctgcaagaaattttacaagacaaagaagatgtgtactcgccggttaa

SEQ ID NO: 35 - protein, 468 aa, Clostridium saccharoperbutylacetonicum N1-4(HMT)
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPGMVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYSINKKWVGKDAKLFLDEIDVESPSSVKCIICEVSARHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG